(19) United States
(12) Patent Application Publication
(10) Pub. No.: US 2003/0004123 A1
(43) Pub. Date: Jan. 2, 2003

(54) Targeted gene transfer using G protein coupled receptors

(76) Inventors: Richard C. Boucher JR., Chapel Hill, NC (US); Raymond J. Pickles, Chapel Hill, NC (US); Janet L. Rideout, Raleigh, NC (US); William Pendergast, Durham, NC (US); Benjamin R. Yerxa, Raleigh, NC (US)

Correspondence Address: MYERS BIGEL SIBLEY & SAJOVEC, PO BOX 37428, RALEIGH, NC, 27627, US

(21) Appl. No.: 10/052,179

(22) Filed: Jan. 17, 2002

Related U.S. Application Data
(63) Continuation of application No. 09/219,698, filed on Dec. 23, 1998, now abandoned.
Application No. 09/219,698 is a continuation-in-part of application No. 09/105,527, filed on Jun. 26, 1998, now abandoned.
(63) Continuation-in-part of application No. 09/446,480, filed on Dec. 21, 1999, now abandoned.
Application No. 09/446,480 is a 371 of international application No. PCT/US98/13336, filed on Jun. 26, 1998.
(60) Provisional application No. 60/050,843, filed on Jun. 26, 1997.
(60) Provisional application No. 60/050,843, filed on Jun. 26, 1997.

(51) Int. Cl. A61K 48/00; C12N 15/87

(52) U.S. Cl. 514/44; 435/455

(57) ABSTRACT

A method of delivering heterologous nucleic acid (e.g., a gene sequence) into a cell comprises attaching a virus containing a heterologous gene sequence to a G protein coupled receptor (i.e., a seven transmembrane receptor such as the P2Y₂ receptor). The virus may be attached to the receptor by means of a bridging antibody, or by binding an antibody specific for the receptor with an antibody specific for the virus, wherein the antibody that specifically binds with the receptor and the antibody that specifically binds to the virus are cross-linked. Alternatively, the virus may express a peptide that specifically binds to the receptor. The receptor may be induced to internalize by means of the addition of a ligand known to trigger internalization of the receptor into the cell.

### RELATED APPLICATION INFORMATION

This application is a continuation-in-part of copending application Ser. No. 09/105,527, filed Jun. 26, 1998, which claims the benefit of U.S. Provisional Application No. 60/050,843 filed Jun. 26, 1997, the disclosures of which are incorporated herein by reference.

### STATEMENT OF FEDERAL SUPPORT

This invention was made with Government support under Grant No. HL51818 from the National Institute of Health. The government has certain rights in this invention.

### FIELD OF THE INVENTION

This invention relates to methods and systems useful in the transfer of nucleic acids into eukaryotic cells.

### BACKGROUND OF THE INVENTION

The capacities to introduce a particular foreign or native gene sequence into a mammalian cell and to control the expression of that gene are of substantial value in the fields of medical and biological research. Such capacities provide a means for studying gene regulation, and for designing a therapeutic basis for the treatment of disease.

The introduction of a particular foreign or native gene into mammalian host cells is facilitated first by introducing a gene sequence into a suitable nucleic acid vector. A variety of methods have been developed which are capable of permitting the introduction of such a recombinant vector into a desired host cell. The use of viral vectors can result in the rapid introduction of the recombinant molecule in a wide variety of host cells. In particular, viral vectors have been employed in order to increase the efficiency of introducing a recombinant nucleic acid vector into host cells. Viruses that have been employed as vectors for the transduction and expression of exogenous genes in mammalian cells include SV40 virus (see, e.g., H. Okayama et al., Molec. Cell. Biol. 5, 1136-1142 (1985)); bovine papilloma virus (see, e.g., D. DiMaio et al., Proc. Natl. Acad. Sci. USA 79, 4030-4034 (1982)); adenovirus (see, e.g., J. E. Morin et al., Proc. Natl. Acad. Sci. USA 84, 4626 (1987)), adeno-associated virus (AAV; see, e.g., N. Muzyczka et al., J. Clin. Invest. 94, 1351 (1994)); herpes simplex virus (see, e.g., A. I. Geller, et al., Science 241, 1667 (1988)), and others.

Efforts to introduce recombinant molecules into mammalian cells have been hampered by the inability of many cells to be infected by the above-described viral or retroviral vectors. Limitations on retroviral vectors, for example, include a relatively restricted host range, based in part on the level of expression of the membrane protein that serves as the viral receptor. M. P. Kavanaugh et al., Proc. Natl. Acad. Sci USA 91, 7071-7075 (1994).

Accordingly, there exists a need in the art for improved methods of introducing and expressing genes in target cells.

### SUMMARY OF THE INVENTION

The shortcomings of current methods of receptor-mediated gene transfer are overcome by the methods and complexes of the present invention. In particular, the invention is based upon the unexpected discovery by the present inventors that the rate limiting step of viral vector uptake by cell surface receptors is not, as originally thought, the binding event of the virus to the receptor, but rather the internalization of the receptor itself. Accordingly, this invention relates to new complexes that facilitate the transfer of nucleic acids into eukaryotic cells. This invention allows for targeting transfer vectors to specific cell types, attachment of the vectors to the cells and regulated cellular internalization of the vectors.

This invention comprises binding a transfer vector to a receptor that is internalized by a cell. The receptor is one that is either internalized by a cell upon the cell's exposure to a specific ligand, or for which a receptor may be induced to internalize by exposure to such a ligand.

### BRIEF DESCRIPTION OF THE DRAWINGS

FIG. 1 is a schematic drawing illustrating a virus-receptor complex of the present invention. This Figure illustrates an adenovirus targeted to an internalizing seven transmembrane receptor.

FIG. 2 is a schematic representation of particular embodiments of the present invention.

FIG. 3 is a graphical representation of the Cl⁻secretory responses of human airway epithelia to lumenal NECA (A_{2b} agonist), isoproterenol, bradykinin, or ATP (all 10⁻⁴ M).

FIG. 4A is a graphical representation of the dose-effect relationship between bs-Ab concentration and gene transfer efficiency in A9-null cells (●) compared with HA-P2Y₂-A9 administered sequentially (∘) or as preformed conjugates (▴).

FIG. 4B is a graphical representation of a study to evaluate the specificity of increased gene transfer with bs-Ab in A9-HA-P2Y₂ and A9-null cells pre-treated with specific or non-specific bs-Ab or after chronic desensitization of HA-P2Y₂ receptors by pretreatment with ATPγS.

FIG. 5 is a graphical representation of gene transfer with bs-Ab in null A9 and A9 cells expressing an HA-tagged BK_{II} receptor.

FIG. 6 is a graphical representation of gene transfer in CHO cells with bs-Ab to HA-tagged P2Y₂ and P2 receptors and adenovirus fiber protein.

FIG. 7 is a graphical representation of biotin-UTP stimulation of inositol phosphate formation in P2Y₂ receptor expressing (●) but not wild-type (▪) astrocytoma cells.

FIG. 8 is a graphical representation of the stimulation of gene transfer in A9 (wt) and HA-P2Y₂-A9 cells in the presence of biotin-UTP conjugated by streptavidin to biotin-Ad.

FIG. 9A is a graphical representation of a comparison of agonist potency of U₂P₄ and UTP in astrocytoma cells expressing P2Y₂ receptors.

FIG. 9B is a graphical representation of the metabolic stability of U₂P₄ compared with UTP in cystic fibrosis sputum.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will fully convey the scope of the invention to those skilled in the art. Like numbers refer to like elements throughout.

A transfer vector-receptor complex of the present invention comprises a transfer vector bound to a receptor that is capable of being internalized into a cell. The transfer vector may contain an exogenous nucleic acid sequence (e.g., a gene), and may express an exogenous protein or peptide. In particular preferred embodiments, described in more detail hereinbelow, the transfer vector is targeted to a seven transmembrane (7-TM) receptor by means of an antibody specific to the receptor, by means of a peptide expressed by the transfer vector that specifically binds said receptor, or by means of a natural or modified ligand. The transfer vector may be any suitable vector, including a viral vector, a plasmid, an oligonucleotide, or RNA/DNA chimeric molecules, as is described more fully hereinbelow. Interaction between the 7-TM receptor and the targeted complex results in receptor-complex internalization, thereby introducing the heterologous nucleic acid carried by the transfer vector into the cell where it is expressed.

A. Viral Transfer Vectors.

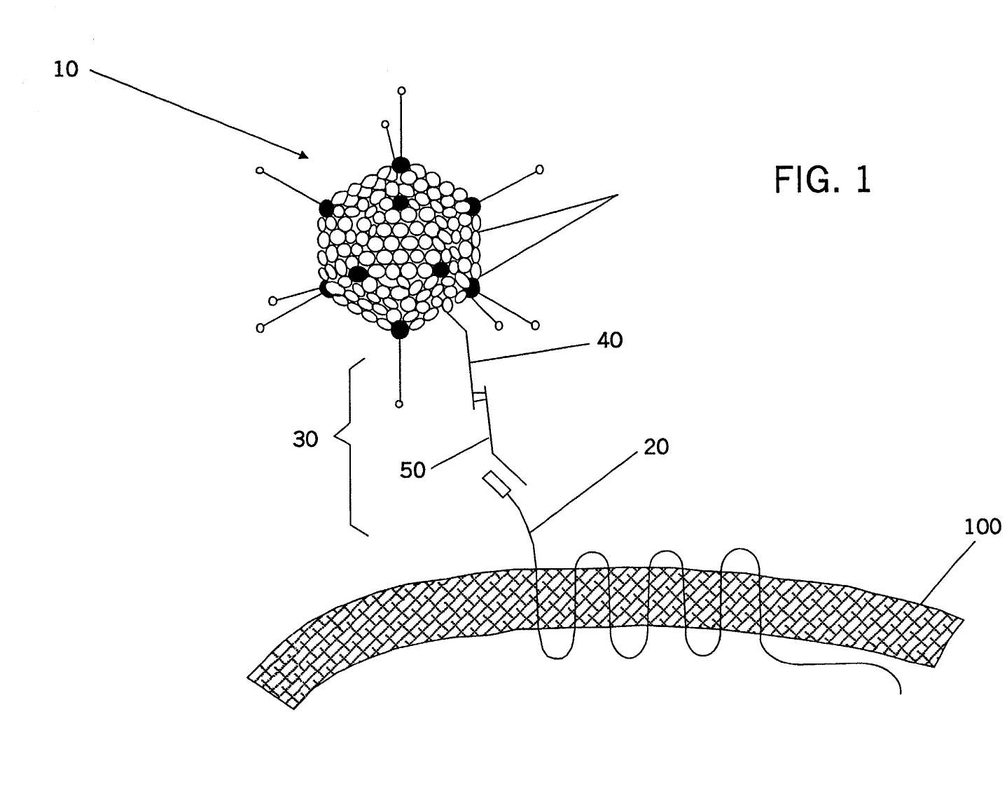

One embodiment of the invention is described with reference to FIG. 1. In this embodiment, a complex (i.e., a conjugate) of the present invention comprises a viral vector 10 (which is illustrated in the figure as an adenovirus) attached to a 7-TM receptor 20, which receptor 20 is present on a cell surface 100. The viral vector 10 is attached to the 7-TM receptor 20 by means of a bifunctional bridging antibody 30. The bifunctional bridging antibody 30 is composed of one antibody 40 which specifically binds the viral vector. The antibody 40 is chemically cross-linked to antibody 50, which specifically binds the 7-TM receptor 20.

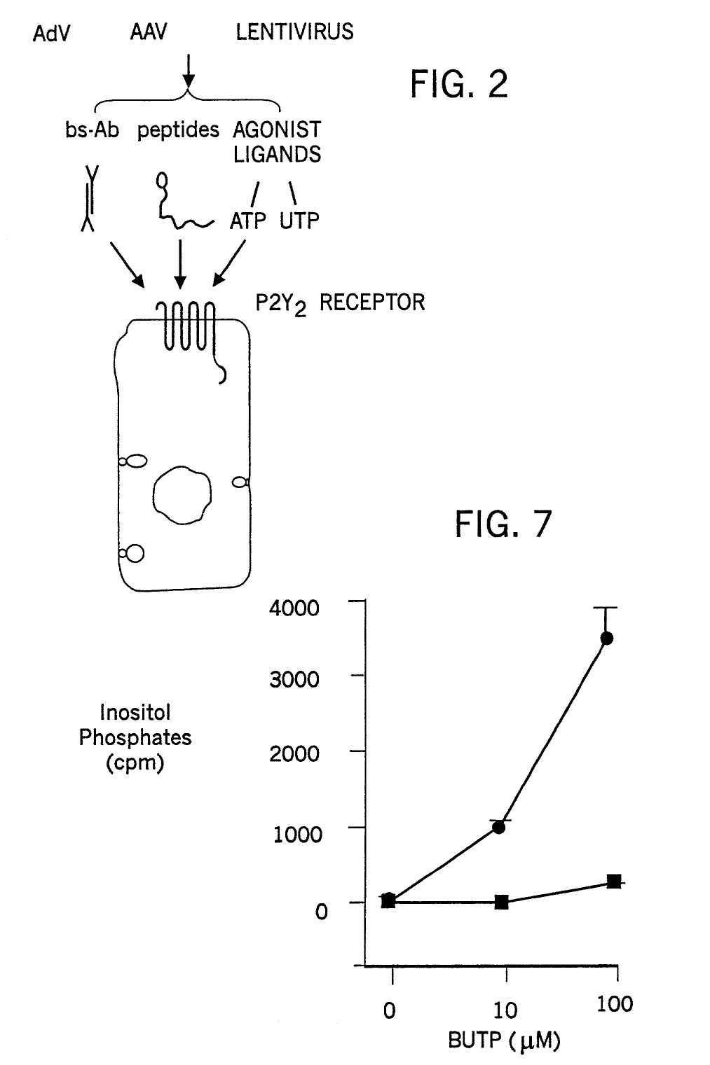

Although the viral vector 10 is illustrated as an adenovirus vector (AdV), it will be understood that the present invention may also be practiced with other viral vectors, including but not limited to human and nonhuman retrovirus (ie., Maloney virus such as Moloney Murine Leukemia Virus and lentiviruses) vectors, adeno-associated virus (AAV) vectors, and herpes virus vectors (FIG. 2). The viral vectors of the present invention may be attenuated viruses or may be rendered non-replicative by any method known to one skilled in the art.

However, the use of adenoviruses as the vector is currently preferred.

The viral vectors of the present invention will have the capacity to include exogenous nucleic acids. The delivery of the heterologous nucleic acid facilitates the replication of the heterologous nucleic acid within the target cell, and the subsequent production of a heterologous protein therein. A heterologous protein is herein defined as a protein or fragment thereof wherein all or a portion of the protein is not naturally expressed by the target cell. A nucleic acid or gene sequence is said to be heterologous if it is not naturally present in the wild type of the viral vector used to deliver the gene into a cell (e.g., the wild-type adenovirus genome). The term “nucleic acid sequence” or “gene sequence, ” as used herein, is intended to refer to a nucleic acid molecule (preferably DNA). Such gene sequences may be derived from a variety of sources including DNA, cDNA, synthetic DNA, RNA or combinations thereof. Such gene sequences may comprise genomic DNA which may or may not include naturally occurring introns. Moreover, such genomic DNA may be obtained in association with promoter sequences or poly-adenylation sequences. The gene sequences of the present invention are preferably cDNA. Genomic or cDNA may be obtained in any number of ways. Genomic DNA can be extracted and purified from suitable cells by means well-known in the art. Alternatively, mRNA can be isolated from a cell and used to prepare cDNA by reverse transcription, or other means.

Standard techniques for the construction of the vectors of the present invention are well-known to those of ordinary skill in the art and can be found in such references as Sambrook et al., Molecular Cloning: A Laboratory Manual 2nd Ed. (Cold Spring Harbor, N.Y., 1989). A variety of strategies are available for ligating fragments of DNA, the choice of which depends on the nature of the termini of the DNA fragments and which choices can be readily made by the skilled artisan.

As will be appreciated by one skilled in the art, the nucleotide sequence of the inserted heterologous gene sequence or sequences may be of any nucleotide sequence. For example, the inserted heterologous gene sequence may be (or may include) a reporter gene sequence or a selectable marker gene sequence. A reporter gene sequence, as used herein, is any gene sequence which, when expressed, results in the production of a protein whose presence or activity can be monitored. Examples of suitable reporter genes include the gene for galactokinase, beta-galactosidase, chloramphenicol acetyltransferase, beta-lactamase, etc. Alternatively, the reporter gene sequence may be any gene sequence whose expression produces a gene product which affects cell physiology.

A selectable marker gene sequence is any gene sequence capable of expressing a protein whose presence permits one to selectively propagate a cell which contains it. Examples of selectable marker genes include gene sequences capable of conferring host resistance to antibiotics (e.g., puromycin, ampicillin, tetracycline, kanamycin, and the like), or of conferring host resistance to amino acid analogues, or of permitting the growth of bacteria on additional carbon sources or under otherwise impermissible culture conditions. A gene sequence may be both a reporter gene and a selectable marker gene sequence. The most preferred reporter genes of the present invention are the lacZ gene which encodes the beta-galactosidase activity of E. coli; and the gene encoding puromycin resistance.

Preferred reporter or selectable marker gene sequences are sufficient to permit the recognition or selection of the vector in normal cells. In one embodiment of the invention, the reporter gene sequence will encode an enzyme or other protein which is normally absent from mammalian cells, and whose presence can, therefore, definitively establish the presence of the vector in such a cell.

The heterologous gene sequence may also comprise the coding sequence of a desired product such as a suitable biologically active protein or polypeptide, immunogenic or antigenic protein or polypeptide, or a therapeutically active protein or polypeptide. Preferably, the heterologous gene sequence encodes a therapeutically active protein or polypeptide. In one particular preferred embodiment, the heterologous gene sequence encodes the cystic fibrosis transmembrane conductance regulator (CFTR) protein or biologically active analogs, fragments, or derivatives thereof. Alternatively, the heterologous gene sequence may comprise a sequence complementary to an RNA sequence, such as an antisense RNA sequence, which antisense sequence can be administered to an individual to inhibit expression of a complementary polynucleotide in the cells of the individual.

Expression of the heterologous gene may provide immunogenic or antigenic protein or polypeptide to achieve an antibody response, which antibodies can be collected from an animal in a body fluid such as blood, serum or ascites.

It is also possible to employ as the inserted heterologous gene sequence a gene sequence which already possesses a promoter, initiation sequence, or processing sequence.

B. Non-Viral Vectors.

In other preferred embodiments, the transfer vector is non-viral. Other suitable vectors include, but are not limited to, oligonucleotides (including RNA, DNA, synthetic and modified nucleic acids), plasmids, and RNA/DNA chimeric molecules as described by E. Kometz. Oligonucleotide vectors include antisense oligonucleotides and oligonucleotides that function as ribozymes. The non-viral transfer vectors of the present invention are able to include exogenous nucleic acids as described hereinabove with respect to viral vectors. Oligonucleotides, plasmids, and RNA/DNA chimeric molecules can be synthesized or produced by any suitable method known in the art.

C. Seven Transmembrane Receptors

Receptors that may be used to carry out the present invention belong to the family of 7-TM receptors. See generally, S. Watson et al., The G-Protein Linked Receptor FactsBook, Academic Press, New York (1994); U.S. Pat. No. 5,482,835 to King et al.

Those skilled in the art will appreciate that 7-TM receptors are G protein coupled receptors. Any mammalian G protein coupled receptor, and the nucleic acid sequences encoding these receptors, may be employed in practicing the present invention. Examples of such receptors include, but are not limited to, dopaaminereceptors, muscarinic cholinergic receptors, cc-adrenergic receptors, opiate receptors, cannabinoid receptors, serotonin receptors, β-adrenergic receptors, and purinoceptors. The term “receptor” as used herein is intended to encompass subtypes of the named receptors, and mutants and homologs thereof, along with the nucleic acid sequences encoding the same.

Preferably, the 7-TM receptor for use according to the present invention is a purinoceptor as discussed in greater detail below (e.g., P2Y₁, P2Y₂, P2Y₄, P2Y₆ and P2Y₁₁), an adenosine receptor (i.e., A1, A2, and A3, and sub-types thereof), a bradykinin receptor (e.g., BK_{I} and BK_{II}), or a β-adrenergic receptor (e.g., β¹, β₂ and β₃). Also preferred is the C_{5A} complement receptor. More preferred are the P2Y₂, BK_{II}, A_{2B}, β₂, and C_{5A} receptors, with the P2Y₂ receptor being most preferred. Thus, ligands that may be used to carry out the present invention include nucleotides, nucleosides, catecholamines (e.g., dopamine, 5-hydroxytryptophan), C5A, and bradykinin(s).

The P2Y₂ (also known as the P_{2U})-purinoceptor undergoes internalization upon activation with ATP, UTP and analogs thereof. These receptor types are abundant in number on the lumenal surface of the human respiratory epithelium. Mason, S. J., et al. 1991. Br. J Phannacol. 103, 1649-1656. Molecular conjugation of AdV to P2Y₂-receptors, followed by activation of these receptors by ATP/UTP, leads to internalization of the vector-ligand-receptor complex into endosomes and thus provide an alternative entry pathway for AdV into the well differentiated (WD) epithelium, and thereafter to gene expression.

D. Antibodies.

As shown in FIG. 1 and FIG. 2, one strategy for targeting transfer vectors carrying heterologous nucleic acids to 7-TM receptors for internalization into the cell is with a bispecific bridging antibody. In general, the bispecific antibody is directed against epitopes on both the transfer vector and the 7-TM receptor of interest (i.e., has a combining region that specifically recognizes the transfer vector and a combining region that specifically recognizes the 7-TM receptor), thereby forming a “bridge” between the transfer vector and the receptor. Binding of the bispecific bridging antibody to the 7-TM receptor induces internalization of the receptor. The bound antibody-transfer vector complex is internalized along with the 7-TM receptor, thereby introducing the transfer vector carrying the heterologous nucleic acid into the cell. According to this embodiment of the invention, the transfer vector is preferably a viral vector, more preferably, AdV, and the bispecific antibody comprises a monoclonal antibody directed against the fiber (knob) protein of the adenovirus.

The term “antibodies” as used herein refers to all types of immunoglobulins, including IgG, IgM, IgA, IgD, and IgE. Of these, IgM and IgG are particularly preferred. The antibodies may be monoclonal or polyclonal and may be of any species of origin, including (for example) mouse, rat, rabbit, horse, or human, or may be chimeric antibodies. See, e.g., M. Walker et al., Molec. Immunol. 26, 403-11(1989). The antibodies may be recombinant monoclonal antibodies produced according to the methods disclosed in Reading U.S. Pat. No. 4,474,893, or Cabilly et al., U.S. Pat. No. 4,816,567. The antibodies may also be chemically constructed by specific antibodies made according to the method disclosed in SegAl et al., U.S. Pat. No. 4,676,980.

Antibodies may be polyclonal or monoclonal, with monoclonal being preferred. In particular embodiments, the antibodies are bridging antibodies that are specific to both the target receptor and the transfer vector. According to this embodiment, the bridging antibody is preferably a monoclonal antibody directed to the adenovirus fiber (knob) protein. Also preferred are monoclonal antibodies and bridging antibodies comprising monoclonal antibodies that are directed to specific epitopes of the 7-TM receptor of interest.

Antibodies that bind to the same epitope (i.e., the specific binding site) that is bound by an antibody to the 7-TM receptor can be identified in accordance with known techniques, such as their ability to compete with labeled antibody to the 7-TM receptor in a competitive binding assay.

Antibody fragments included within the scope of the present invention include, for example, Fab, F(ab′)2, and Fc fragments, and the corresponding fragments obtained from antibodies other than IgG. Such fragments can be produced by known techniques.

Polyclonal antibodies used to carry out the present invention may be produced by immunizing a suitable animal (e.g., rabbit, goat, etc.) with an antigen to which a monoclonal antibody to the 7-TM receptor binds, collecting immune serum from the animal, and separating the polyclonal antibodies from the immune serum, in accordance with known procedures.

Monoclonal antibodies used to carry out the present invention may be produced in a hybridoma cell line according to the technique of Kohler and Milstein, Nature 265, 495-97 (1975). For example, a solution containing the appropriate antigen may be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable media and the supernatant screened for monoclonal antibodies having the desired specificity. Monoclonal Fab fragments may be produced in Escherichia coli by recombinant techniques known to those skilled in the art. See, e.g., W. Huse, Science 246, 1275-81 (1989).

Antibodies specific to the 7-TM (e.g., P2Y₂) receptor may also be obtained by phage display techniques known in the art.

Those skilled in the art will be familiar with numerous specific immunoassay formats and variations thereof which may be useful for carrying out the method disclosed herein. See generally E. Maggio, Enzyme-Immunoassay, (1980)(CRC Press, Inc., Boca Raton, FL); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” Applicants specifically intend that the disclosures of all U.S. Patent references cited herein be incorporated herein by reference in their entirety.

Antibodies as described herein may be conjugated to a solid support suitable for a diagnostic assay (e.g., beads, plates, slides or wells formed from materials such as latex or polystyrene) in accordance with known techniques, such as precipitation. Antibodies as described herein may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques. The term “antigenic equivalents” as used herein, refers to proteins or peptides which bind to an antibody which binds to the protein or peptide with which equivalency is sought to be established. Antibodies which are used to select such antigenic equivalents are referred to as “selection antibodies” herein.

E. Non-Antibody Based Targeting Strategies

The invention has been described above with respect to bispecific bridging antibodies as means of targeting the transfer vector to the 7-TM receptor for internalization. As shown in FIG. 2, alternative targeting strategies include those utilizing peptides and 7-TM receptor agonist/antagonists.

With respect to peptides, the peptide can be a natural ligand that binds to the 7-TM receptor. Peptide agonists and antagonists of 7-TM receptors are known in the art. Additionally, novel 7-TM receptor agonists/antagonists can be identified as described by U.S. Pat. No. 5,482,835 to King et al. Alternatively, the peptide can be identified by phage display techniques, or any other method in the art, as binding to the 7-TM receptor.

Methods of synthesizing or producing peptides are well-known in the art. In one particular embodiment, nucleic acids encoding the peptide are fused to or inserted into the gene encoding the AdV knob protein, such that a knob-peptide chimeric protein is expressed. It is known, for example, that exogenous nucleic acid can be expressed in the C-terminus or the HI loop region of the knob protein. In alternate embodiments, concatamers of the peptide are expressed in the knob protein.

As a further alternative, targeting can be achieved with peptides incorporated into “receptorbodies”. In general, a receptorbody is a truncated receptor in which a peptide that binds to a 7-TM receptor is substituted for the intramembrane and intracellular region of the adenoviral receptor. This means that the external portion of the viral receptors acts as an “antibody” to the binding region of the virus (it replaces the need for an antibody to AdV). For example, the external domain of the AdV receptor can be fused to a peptide ligand for a 7-TM receptor. This complex will bind to a recombinant AdV so displaying bradykinin peptide on the surface of the virus. The peptide ligand fused to the truncated AdV receptor will then target this complex to the peptide ligand's cognate receptor on the cell surface. As yet a further alternative, the transfer vector can be targeted to the 7-TM receptor by a chemically-linked high affinity agonist/antagonist of the 7-TM receptor. High-affinity agonists/antagonists may be peptide ligands, as described above or are other molecules such as nucleotides (e.g., ATP, UTP), dinucleotides (described in more detail hereinbelow), and derivatives thereof. In addition, P2Y receptor ligands, particularly P2Y₂ receptor ligands, that can be used to carry out the present invention include all of the compounds, particularly the nucleotides and dinucleotides that are P2Y₂ ligands and are disclosed in W. Pendergast et al., U.S. Pat. No. 5,837,861 (Nov. 17, 1998), along with all the compounds disclosed in U.S. Pat. Nos. 5,763,447 to Jacobus and Leighton, 5,789,391 to Jacobus et al., 5,635,160 to Stutts et al., and 5,292,498 to Boucher, the disclosures of all of which are incorporated herein by reference in their entirety.

Examples of such nucleotides are depicted in Formulae I - IV

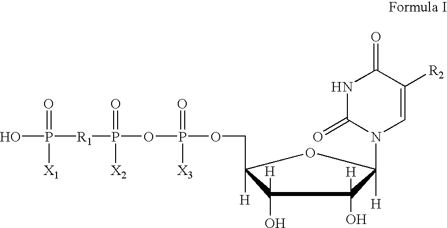

wherein:

X₁, X₂ and X₃ are each independently either O⁻or S⁻; preferably, X₂ and X₃ are O;

R₁ is O, imido, methylene or dihalomethylene (e.g., dichloromethylene or difluoromethylene); preferably, R₁ is oxygen or difluoromethylene;

R₂ is H or Br; preferably, R₂ is H; particularly preferred compounds of Formula I are uridine 5′-triphosphate (UTP) and uridine 5′-O-(3-thiotriphosphate) (UTPγS).

A dinucleotide is depicted by the general Formula II:

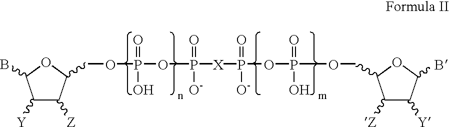

wherein:

X is oxygen, methylene, difluoromethylene, imido;

n=0, 1, or 2;

m=0, 1, or 2;

n+m=0,1,2,3, or 4; and

B and B′ are each independently a purine residue or a pyrimidine residue linked through the 9- or 1-position, respectively;

Z=OH or N₃;

Z′=OH or N₃;

Y=H or OH;

Y′=H or OH;

provided that when Z is N₃, Y is H or when Z′ is N₃, Y′ is H.

The furanose sugar is preferably in the β-configuration.

The furanose sugar is most preferably in the β-D-configuration.

Preferred compounds of Formula II are the compounds of Formula IIa:

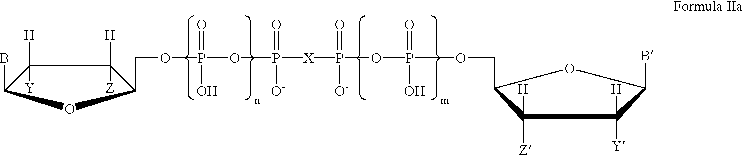

wherein:

X=O;

n+m=1 or 2;

Z, Z′, Y, and Y′=OH;

B and B′ are defined in Formulas IIc and IId;

X=O;

n+m=3 or 4;

Z, Z′, Y, and Y′=OH;

B=uracil;

B′ is defined in Formulas IIc and IId; or

X=O;

n+m=1 or 2;

Z, Y, and Y′=OH;

Z′=H;

B=uracil;

B′ is defined in Formulas IIc and IId; or

X=O;

n+m=0, 1, or 2;

Z and Y=OH;

Z′=N₃;

Y′=H;

B=uracil;

B′=thymine; or

X=O;

n+m=0, 1, or 2;

Z and Z′=N₃;

Y and Y′=H;

B and B′=thymine; or

X=CH₂, CF₂, or NH;

n and m=1;

Z, Z′, Y, and Y′=OH;

B and B′ are defined in Formulas IIc and IId.

Another preferred group of the compounds of Formula II are the compounds of Formula IIb or the pharmaceutically acceptable salts thereof:

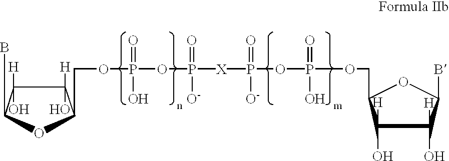

wherein:

X is oxygen, methylene, difluoromethylene, or imido;

n=0 or 1;

m=0 or 1;

n+m=0, 1, or 2; and

B and B′ are each independently a purine residue, as in Formula IIc, or a pyrimidine residue, as in Formula IId, linked through the 9- or 1-position, respectively. In the instance where B and B′ are uracil, attached at N-I position to the ribosyl moiety, then the total of m +n may equal 3 or 4 when X is oxygen. The ribosyl moieties are in the D-configuration, as shown, but may be L-, or D- and L-. The D-configuration is preferred.

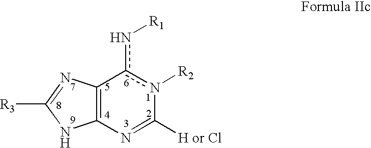

The substituted derivatives of adenine include adenine 1-oxide; 1,N6-(4- or 5-substituted etheno) adenine; 6-substituted adenine; or 8-substituted aminoadenine, where R′ of the 6- or 8-HNR′ groups are chosen from among: arylalkcyl (C₁₋₆) groups with the aryl moiety optionally functionalized as described below; alkyl; and alkyl groups with functional groups therein, such as: ([6-aminohexyl]carbamoylmethyl)-, and ω-acylated-amino(hydroxy, thiol and carboxy) derivatives where the acyl group is chosen from among, but not limited to, acetyl, trifluroroacetyl, benzoyl, substituted-benzoyl, etc., or the carboxylic moiety is present as its ester or arnide derivative, for example, the ethyl or methyl ester or its methyl, ethyl or benzamido derivative. The ω-amino(hydroxy, thiol) moiety may be alkylated with a C₁₋₄ alkyl group.

Likewise, B or B′ or both in Formula IIb may be a pyrimidine with the general formula of Figure IId, linked through the 1-position:

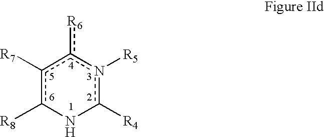

wherein:

R₄ is hydroxy, mercapto, amino, cyano, aralkoxy, C₁₋₆ alkoxy, C₁₋₆ alkylamino, and dialkylamino, the alkyl groups optionally linked to form a heterocycle;

R₅ is hydrogen, acyl, C₁₋₆ alkyl, aroyl, C₁₋₅ alkanoyl, benzoyl, or sulphonate;

R₆ is hydroxy, mercapto, alkoxy, aralkoxy, C₁₋₆-alkylthio, C₁₋₅ disubstituted amino, triazolyl, alkylamino, or dialkylamino, where the alkyl groups are optionally linked to form a heterocycle or linked to N-3 to form an optionally substituted ring;

R₇ is hydrogen, hydroxy, cyano, nitro, alkenyl, with the alkenyl moiety optionally linked through oxygen to form a ring optionally substituted on the carbon adjacent to the oxygen with alkyl or aryl groups, substituted alkynyl or hydrogen where R₈ is amino or substituted amino and halogen, alkyl, substituted alkyl, perhalomethyl (e.g., CF₃), C₂₋₆ alkyl, C₂₋₃ alkenyl, or substituted ethenyl (e.g., alkylamino, bromvinyl and ethyl propenoate, or propenoic acid), C₂₋₃ alkynyl or substituted alkynyl when R₆ is other than amino or substituted amino and together R₅-R₆ may form a 5- or 6-membered saturated or unsaturated ring bonded through N or O at R₆, such a ring may contain substituents that themselves contain functionalities;

R₈ is hydrogen, alkoxy, arylalkoxy, alkylthio, arylalkylthio, carboxamidomethyl, carboxymethyl, methoxy, methylthio, phenoxy, or phenylthio.

In the general structure of Figure IId above, the dotted lines in the 2- to 6-positions are intended to indicate the presence of single or double bonds in these positions; the relative positions of the double or single bonds being determined by whether the R₄, R₆, and R₇ substituents are capable of keto-enol tautomerism.

In the general structures of Figure IIc and IId above, the acyl groups advantageously comprise alkanoyl or aroyl groups. The alkyl groups advantageously contain 1 to 8 carbon atoms, particularly 1 to 4 carbon atoms optionally substituted by one or more appropriate substituents, as described below. The aryl groups including the aryl moieties of such groups as aryloxy are preferably phenyl groups optionally substituted by one or more appropriate substituents, as described below. The above mentioned alkenyl and alkynyl groups advantageously contain 2 to 8 carbon atoms, particularly 2 to 6 carbon atoms, e.g., ethenyl or ethynyl, optionally substituted by one or more appropriate substituents as described below. Appropriate substituents on the above-mentioned alkyl, alkenyl, alkynyl, and aryl groups are advantageously selected from halogen, hydroxy, C₁₋₄ alkoxy, C₁₋₄ alkyl, C₆₋₁₂ arylalkoxy, carboxy, cyano, nitro, sulfonamido, sulfonate, phosphate, sulfonic, amino, and substituted amino wherein the amino is singly or doubly substituted by a C₁₋₄ alkyl, and when doubly substituted, the alkyl groups optionally being linked to form a heterocycle.

For purposes of further clarifying the foregoing descriptions of Formulae IIc and IId, the descriptions can be simplified to the following:

R₂ is O or is absent; or

R₁ and R₂ taken together may form optionally substituted 5-membered fused imidazole ring; or

R₁ of the 6-HNR₁ group or R₃ of the 8-HNR₃ group is chosen from the group consisting of:

(a) arylalkyl (C₁₋₆) groups with the aryl moiety optionally substituted,

(b) alkyl,

(c) ([6-aminohexyl]carbamoylmethyl),

(d) ω-amino alkyl (C₂₋₁₀),

(e) ω-hydroxy alkyl (C₂₋₁₀),

(f) ω-thiol alkyl (C₂₋₁₀),

(g) ω-carboxy alkyl (C₂₋₁₀),

(h) the ω-acylated derivatives of (b), (c) or (d) wherein the acyl group is either acetyl, trifluroacetyl, benzoyl, or substituted-benzoyl alkyl(C₂₋₁₀), and

(i) ω-carboxy alkyl (C₂₋₁₀) as in (e) above wherein the carboxylic moiety is an ester or an amide;

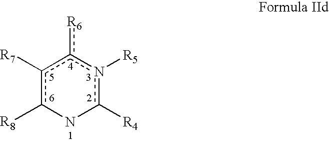

wherein:

R₄ is hydroxy, mercapto, amino, cyano, aralkoxy, C₁₋₆ alkylthio, C₁₋₆ alkoxy, C₁₋₆ alkylamino or dialkylamino, wherein the alkyl groups of said dialkylamino are optionally linked to form a heterocycle;

R₅ is hydrogen, acyl, C₁₋₆ alkyl, aroyl, C₁₋₅ alklanoyl, benzoyl, or sulphonate;

R₆ is hydroxy, mercapto, alkoxy, aralkoxy, C₁₋₆-alkylthio, C₁₋₅ disubstituted amino, triazolyl, alkylamino or dialkylamino, wherein the alkyl groups of said dialkylamino are optionally linked to form a heterocycle or linked to N³ to form an optionally substituted ring;

R₅-R₆ together forms a 5 or 6-membered saturated or unsaturated ring bonded through N or O at R₆, wherein said ring is optionally substituted;

R₇ is selected from the group consisting of:

(a) hydrogen,

(b) hydroxy,

(c) cyano,

(d) nitro,

(e) alkenyl, wherein the alkenyl moiety is optionally linked through oxygen to form a ring optionally substituted with alkyl or aryl groups on the carbon adjacent to the oxygen,

(f) substituted alkynyl

(g) halogen,

(h) alkyl,

(i) substituted alkyl,

(j) perhalomethyl,

(k) C₂₋₆ alkyl,

(l) C₂₋₃ alkenyl,

(m) substituted ethenyl,

(n) C₂₋₃ alkynyl and

(o) substituted alkynyl when R is other than amino or substituted amino;

R₈ is selected from the group consisting of:

(a) hydrogen,

(b) alkoxy,

(c) arylalkoxy,

(d) alkylthio,

(e) arylalkylthio,

(f) carboxamidomethyl,

(g) carboxymethyl,

(h) methoxy,

(i) methylthio,

(j) phenoxy and

(k) phenylthio.

CTP and its analogs are depicted by general Formula III:

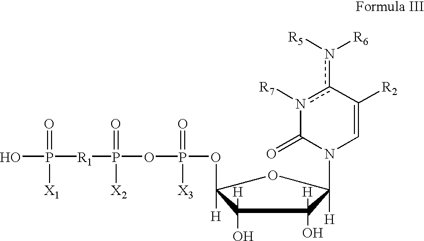

wherein:

R₁, X₁, X₂ and X₃ are defined as in Formula I;

R₅ and R₆ are H while R₇ is nothing and there is a double bond between N-3 and C-4 (cytosine), or

R₅, R₆ and R₇ taken together are —CH═CH—, forming a ring from N-3 to N-4 with a double bond between N-4 and C-4 (3,N⁴-ethenocytosine) optionally substituted at the 4- or 5-position of the etheno ring.

ATP and its analogs are depicted by general Formula IV:

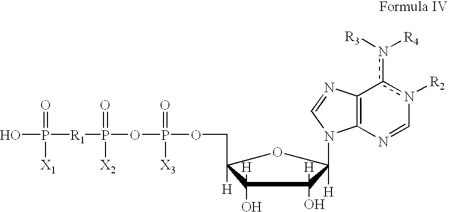

wherein:

R₁, X₁, X₂, and X₃ are defined as in Formula I;

R₃ and R₄ are H while R₂ is nothing and there is a double bond between N-1 and C-6 (adenine), or

R₃ and R₄ are H while R₂ is O and there is a double bond between N-1 and C-6 (adenine 1-oxide), or

R₃, R₄, and R₂ taken together are —CH═CH—, forming a ring from N-6 to N-1 with a double bond between N-6 and C-6 (1,N6-ethenoadenine).

For simplicity, Formulas I, II, III, and IV herein illustrate the active compounds in the naturally occurring D-configuration, but the present invention also encompasses compounds in the L-configuration, and mixtures of compounds in the D- and L-configurations, unless otherwise specified. The naturally occurring D-configuration is preferred.

Some compounds of Formulas I, II, III, and IV can be made by methods which are well known to those skilled in the art and in accordance with known procedures (Zamecnik, P., et al., Proc. Natl Acad. Sci. USA 89:2370-2373 (1992); Ng, K., et al., Nucleic Acids Res. 15:3572-3580 (1977); Jacobus, K.M., et al., U.S. Pat. No. 5,789,391 and Pendergast, W., et al., International Patent Application WO98/34942)); some are commercially available, for example, from Sigma Chemical Company, PO Box 14508, St. Louis, Mo. 63178. The synthetic methods of U.S. Pat. No. 5,789,391 and International Patent Application WO98/34942 are incorporated herein by reference.

In a further alternative embodiment, high affinity agonists or antagonists can be directly linked to the transfer vector using sulfo-N-hydroxysuccinimide (NHS) as described in more detail below.

F. Biotin and Covalent Conjugates

Examples of compounds that can be used to carry out the present invention include compounds of Formula I-IV above, and include compounds having the general formula:

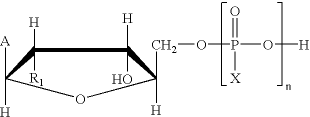

wherein:

X may be O or S;

A is a purine or pyrimidine base (e.g., adenine, guanine, thymine, cytosine, uracil)(each purine or pyrimidine base is preferably joined to the ribose or deoxyribose ring by covalent bond to the 9 nitrogen in the case of purines, or by covalent bond to the 1 nitrogen in the case of pyrimidines);

R₁ is H or OH; and

n is from 1 to 4 or 6, preferably 2, 3 or 4.

The transfer vector is covalently or noncovalently joined or conjugated to the purine or pyrimidine base, or the corresponding ribose or deoxyribose ring (e.g., of the compounds of Formula I-IV above), or attached to the terminal phosphate moiety of compounds represented by Formulae I, II and IV above, by any suitable means, such as by covalently joining a linking chain (e.g., a linking polymer chain) thereto in any suitable position (e.g., a ring carbon such as the 5 carbon in a pyrimidine, or the 2, 6 or 8 carbon in a purine), to which linking group the ligand may be covalently attached, or to which linking group a biotin group may be attached, with a biotin group covalently joined to the ligand (see below) and the two biotin groups joined to one another by means of an avidin group to which both biotin groups are joined or conjugated.

Specific examples of ligands that can be used to carry out the present invention include those having the general formula:

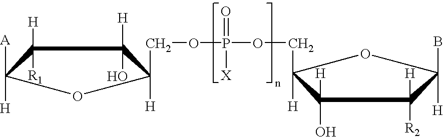

wherein:

X maybe O or S;

A and B are each independently a purine or pyrimidine base (e.g., adenine, guanine, thymine, cytosine, uracil); preferably, one of A or B is uracil; in one particularly preferred embodiment, A is uracil and B is cytosine;

R₁ and R₂ are each independently selected from the group consisting of H or OH;

n is from 1 to 4 or 6, preferably 2, 3 or 4; and

said transfer vector is covalently or noncovalently conjugated or joined to A or B or the ribose or deoxyribose ring -to which A or B is joined, either directly or indirectly by means of a linking group, in the same manner as described above.

In one particular embodiment, a biotin (B)-UTP is used as a targeting agonist for the P2Y₂ receptor. The B-UTP can interact with a biotinylated viral transfer vector in the presence of streptavidin (SA) to give a virus-biotin-SA-biotin-UTP complex that will be targeted to the P2Y₂ receptor. Alternatively, oligonucleotides, plasmids, and RNA/DNA chimeric molecules can be synthesized or produced to incorporate B-UTP or any other suitable labeled nucleotide.

A particular embodiment of a biotin-dinucleotide conjugate is the structure represented by the formula:

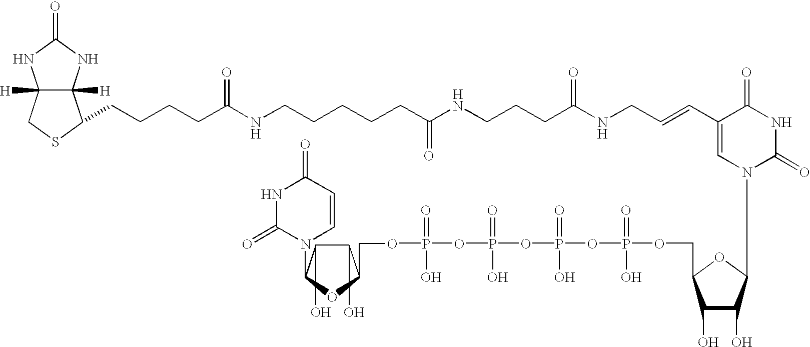

wherein the biotinyl moiety is linked to the pyrimidine base by an aminoallyl linker;

another particular embodiment of a biotin-dinucleotide conjugate is represented by the formula:

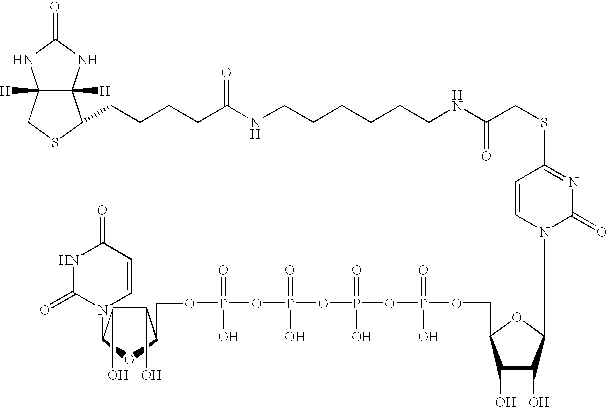

wherein the biotinyl moiety is linked to the pyrimidine base by a linker attached through alkylation of a thiol group

Alternatively, P2Y ligand conjugates may be formed by linking a biotinyl moiety or linking the vector directly through an etheno moiety fused to a purine or pyrimidine base.

Particular embodiments of etheno-linked conjugates are structures of the general formulas for optionally substituted ethenocytidine and ethenoadenosine triphosphates and analogs thereof:

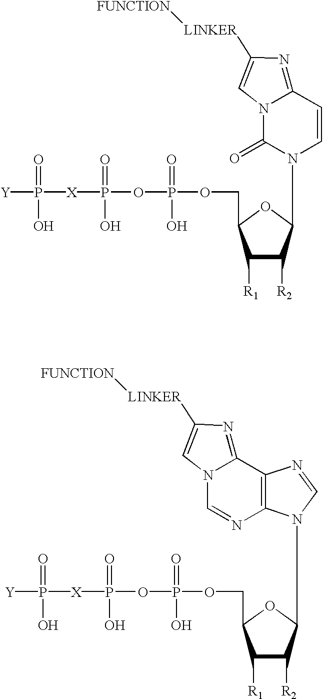

Wherein:

LINKER represents any straight chain of 2-24 atoms in length, or aromatic (e.g. phenyl, naphthyl), or heterocyclic rings (e.g. benzothiophene, isoxazole, pyridine, piperidine) optionally substituted and covalently linking the etheno moiety to the FUNCTION moiety;

FUNCTION represents any of the methods described herein useful to introduce a heterologous nucleic acid into a cell through internalization of the receptor-ligand complex;

R₁ and R₂ may be H, or OH;

X is CH₂, CCl₂, CF₂, NH or O;

Y is O or S.

Other preferred embodiments of etheno-linked conjugates are ethenocytidine and ethenoadenosine dinucleotide compounds of formulas below, wherein LINKER may be attached to the 4- or 5-position of the etheno moiety:

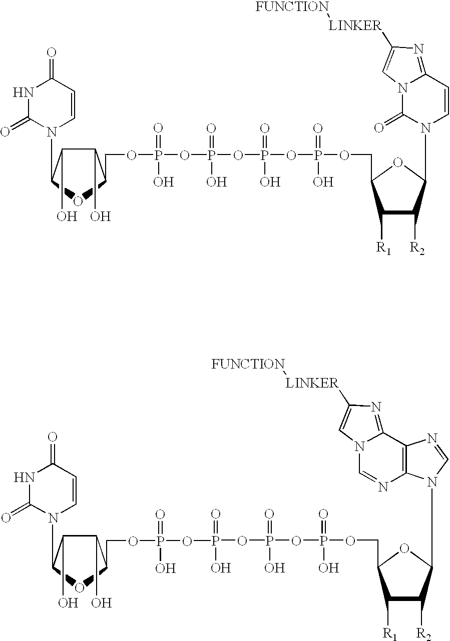

Wherein:

LINKER represents any straight chain of 2-24 atoms in length, or aromatic (e.g. phenyl, naphthyl), or heterocyclic rings (e.g. benzothiophene, isoxazole, pyridine, piperidine) optionally substituted and covalently linking the etheno moiety to the FUNCTION moiety;

FUNCTION represents any of the methods described herein useful to introduce a heterologous nucleic acid into a cell through internalization of the receptor-ligand complex;

R₁ and R₂ may be H, or OH;

Additionally preferred compounds are 6 and 8-substituted adenosine triphosphates and their dinucleotides as described in Formula II.

Links to the carbons of heterocyclic bases may also be made through photoactivatable linkers, such as, but not limited to the use of the commercially available azidophenyl biotin derivative (Pierce Biochemicals, Immunopure Photoactivatable Biotin).

Alternatively, P2 nucleotide ligand conjugates may be formed by attaching a biotinyl moiety through a LINKER or by linking the vector directly through a LINKER to the sugar moiety of the nucleotide, either by derivatization (e.g. acylation, alkylation) of the sugar hydroxyl groups, or through a heteroatom surrogate (e.g. SH, NHR) of the hydroxy groups of molecules represented by formulae I-IV above.

Also P2 nucleotide ligand conjugates may be formed by attaching a biotinyl moiety through a LINKER or by linking the vector directly through a LINKER to the terminal phosphate moiety of molecules represented by formulae I, III and IV above.

Linking groups used to carry out the present invention are, in general, polymers, including both water soluble polymers and water insoluble polymers. Water soluble, or hydrophilic, linking groups are preferred. The polymers are elongate flexible chains of repeating monomeric units, and may carry or contain functional groups along the chain length thereof. Numerous polymers that can be functionalized to function as linking groups for the ligand and the vector, typically by a covalent bond, are known, and will be readily apparent to those skilled in the art. Examples include, but are not limited to, polysaccharides such as dextran, polyvinyl alcohol, polypeptides such as polylysine, and polyacrylic acid. The ligand and the vector may be bound to the linking group in any conformation or position, including to the free chain end thereof In general, the linking group will comprise a chain of from 2 to 24 atoms such as carbon atoms, optionally substituted as described above.

Biotin can be covalently joined to the ligand by conventional techniques and both biotin groups joined to an avidin or streptavidin group in accordance with known techniques to form a conjugate of the vector and the ligand. Examples of ligands to which biotin is covalently joined include:

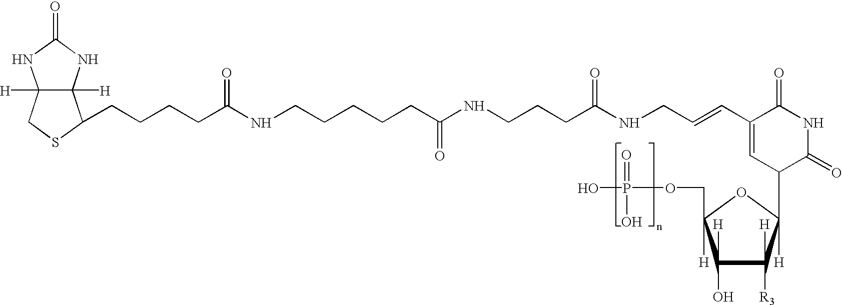

wherein R₂ is H or OH, preferably OH; and n is equal to 1 to 4, preferably 2 or 3. Such compounds are known and commercially available. The uracil group shown can be replaced with another purine or pyrimidine base as described above, with the biotin and linking polymer chain shown between the biotin group and the uridine group above covalently joined to the purine or pyrimidine base in any suitable position (e.g., a ring carbon such as the 5 carbon in a pyrimidine, or the 2 or 8 carbon in a purine). Significantly, an oligonucleotide (e.g., a DNA, RNA, or chimera of 5 or 10 to 30 or 50 bases) can be synthesized with one or more bases conjugated to a biotin in this manner, and the thus biotinylated oligonucleotide conjugated to a biotinylated ligand as described herein by means of an avidin.

A biotin group can be covalently joined to the vector (particularly vectors having free amine groups such as viral vectors) by means of the EZ-LINK™ Sulfo-NHS-LC-Biotinylation Kit, available from Pierce (3747 N. Meridian Road, P.O. Box. 117, Rockford, Ill. 61105). An example of a compound that can be used to biotinylate a primary amine on the vector is Sulfo-NHS-LC-Biotin, available from Pierce, and having the structure:

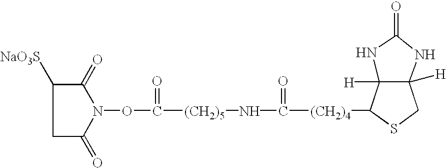

In an alternate embodiment, the biotin group shown in the sulfo compound described above can be removed and replaced with a covalent linkage to a ligand, as described above, to provide a direct covalent linkage from the vector to the ligand.

G. Target Cells

Hematopoietic stem cells, lymphocytes, vascular endothelial cells, respiratory epithelial cells, keratinocytes, skeletal and cardiac muscle cells, neurons and cancer cells are among proposed targets for therapeutic gene transfer, either ex vivo or in vivo. See, e.g., A. D. Miller, Nature 357, 455-460 (1992); R. C. Mulligan, Science 260, 926-932 (1993). These cells and other eukaryotic cells are suitable target cells for the vectors and methods of the present invention. One advantage of the present invention is that it can be used to target heterologous nucleic acids to cells that do not usually bind the transfer vector, i.e, a virus vector.

In particular, any cell that expresses a receptor from the 7-TM receptor family is a suitable target for use according to the present invention. Preferred are cells that express purinoceptors (e.g., P2Y₁, P2Y₂, P2Y₄, P2Y₆, P2Y₁₁), adenosine receptors (i.e., A1, A2, A3), bradykinin receptors (e.g., BK_{I}, BK_{II}), Pβ-adrenergic receptors (e.g., β₁, β₂, β₃)or the C_{SA} complement receptor. More preferred are cells that express P2Y₂, BK_{II}, A_{2B}, β₂, C_{5A} receptors, with cells that express the P2Y₂ receptor being most preferred. Also preferred as targets are respiratory epithelial cells, particularly differentiated columnar airway epithelial cells. The cells may be administered the conjugate in vitro or in vivo, such as by administration of an aerosol containing the conjugate to the luminal surface of airway epithelial cells. Thus purinoceptors that may be used in accordance with the present invention include P2 purinoceptors. Numerous P2 purinoceptors are known. See, e.g., P2 Purinoceptors: Localization, Function and Transduction Mechanisms D. Chadwick and J. Goode Eds 1996. P2 purinoceptors include P2X, P2Y, P2T, P2U (now designated P2Y₂) and P2Z purinoceptors (including subclasses thereof). More recently, the purinoceptors have been classified as a P2Y family, consisting of G protein-mediated receptors, and a P2X family, consisting of ligand-gated cation channels. The P2Z purinoceptors, which open non-selective pores, may be considered a third family. Id. at 6-7. Since different purinoceptors are found on different tissues, the tissue or tissues to be transformed may be determined in part by selection of the purinoceptor to be targeted, and a corresponding ligand selected for the purinoceptor to be targeted, as discussed in greater detail below. Of course, it will be appreciated that many ligands bind to multiple receptor subtypes.

P2X₁ receptors may be used to target vas deferens (e.g., for production of immunocontraceptive vaccines). Ligands that may be used for this receptor include, but are not limited to, 2-methylthio ATP (2-MeSATP), ATP, and α, β-methylene ATP (α,β-meATP).

Immune system cells, including but not limited to monocytes, macrophages, mast cells, neutrophils and B cells, may be targeted with P2Y and/or P2U receptor ligands, as well as P2Z receptor ligands. Such cells may be transformed with a nucleic acid that expresses an immunogen effective to produce an immune response to an antigen or disease vector in the host subject.

Pancreatic cells, such as pancreatic B cells, may be targeted with P2Y receptor ligands. Such cells may be transformed with a nucleic acid that expresses insulin in the cells in an amount effective to combat hypoglycemia or diabetes in the subject.

P2X₃, P2X₄, P2X₅, and P2X₆ receptors may be used to target nerve tissue. Ligands that may be used for these receptors include, but are not limited to, 2-MeSATP, ATP, and (for P2X₃ and P2X₄) α,β-meATP.

P2Z receptors may be used to target macrophages. Ligands that may be used for these receptors include, but are not limited to, 3′-O-(4-benzoyl)benzoyl ATP (BzATP), ATP, and UTP.

P2Y₁ receptors may be used to target nerve tissue, placenta and endothelial tissue. Ligands that may be used for this receptor include, but are not limited to, 2-MeSATP, ATP, ADP, and UTP.

P2Y₂ receptors may be used to target lung, bone, and pituitary tissue. Ligands include, but are not limited to, ATP, UTP, and 2-MeSATP.

P2Y₃ receptors may be used to target nerve tissue. Ligands include, but are not limited to, ADP, UTP, ATP and UDP.

P2Y₄ receptors may be used to target placenta and nerve tissue. Ligands include, but are not limited to, UTP, UDP, ATP and ADP.

P2Y₅ receptors may be used to target lymphocytes. Ligands include, but are not limited to, ADP, ATP and UTP.

P2Y₆ receptors may be used to target blood vessels, particularly smooth muscle and arterial smooth muscle tissue. Ligands include, but are not limited to, UTP, ADP, 2-MeSATP and ATP.

Additional examples of ligands for P2 purinoceptors that may be used to carry out the present invention (and the corresponding receptor subtypes to which they bind) include the P2 purinoceptor antagonists, such as:

quinidine (P2X and P2Y receptors);

imidazolines such as phentolamine (P2Y receptors);

2,2′-pyridylisatogen tosylate, or “PIT” (P2Y receptors);

3-O-3 [N-(4-azido-2-nitrophenyl)amino]proprionyl ATP, or “ANAPP3” (P2X receptors);

Apamin (P2Y receptors);

α,β-meATP (P2X receptors);

reactive blue 2 (P2Y, P2X and P2Z receptors);

suramin (P2X, P2Y, P2T and P2Z receptors);

8-β,5-dinitrophenylene carbonylimino)-1,3,5-napthalene trisulfonate, or “XAMR0721” (P2Y receptors);

pyridoxalphosphate-6-azophenyl-2′,4′-disulfonic acid, or “PPADS” (P2X and P2Y receptors);

pyridoxalphosphate-6-azophenyl-2′,5′disulfonic acid, or “isoPPADS” (P2X receptors);

pyridoxal-5-phosphate, or “P5P” (P2X receptors);

4,4′-diisothiocyanotostilbene-2,2′-disulfonate, or “DIDS” ((P2X receptors);

Evans blue, Trypan blue, and Congo red (P2X receptors);

Brilliant blue (P2Z receptors);

oxidized ATP, or “o-ATP” (P2Z receptors, for targeting macrophages); 2-propylthio-D-β,γ-difluoromethylene ATP, or “ARL 66096” (P2T receptors, for targeting platelets).

H. Gene Transfer

The methods of the present invention provide a means for delivering heterologous nucleic acid independent of the target cell nucleus into a broad phylogenetic range of host cells. The vectors, methods and pharmaceutical formulations of the present invention are additionally useful in a method of administering a protein or peptide to a subject in need of the desired protein or peptide, as a method of treatment or otherwise. In this manner, the protein or peptide may thus be produced in vivo in the subject. The subject may be in need of the protein or peptide because the subject has a deficiency of the protein or peptide, or because the production of the protein or peptide in the subject may impart some therapeutic effect, as a method of treatment or otherwise, and as explained further below.

The gene transfer technology of the present invention has several applications. The most immediate applications are perhaps in elucidating the processing of peptides and functional domains of proteins. Cloned cDNA or genomic sequences for proteins can be introduced into different cell types in culture, or in vivo, in order to study cell-specific differences in processing and cellular fate. By placing the coding sequences under the control of a strong promoter, a substantial amount of the desired protein can be made. Furthermore, the specific residues involved in protein processing, intracellular sorting, or biological activity can be determined by mutational change in discrete residues of the coding sequences.

Gene transfer technology of the present invention can also be applied to provide a means to control expression of a protein and to assess its capacity to modulate cellular events. Some functions of proteins, such as their role in differentiation, may be studied in tissue culture, whereas others will require reintroduction into in vivo systems at different times in development in order to monitor changes in relevant properties.

Gene transfer provides a means to study the nucleic acid sequences and cellular factors which regulate expression of specific genes. One approach to such a study would be to fuse the regulatory elements to be studied to reported genes and subsequently assaying the expression of the reporter gene.

Gene transfer also possesses substantial potential use in understanding and providing therapy for disease states. There are a number of inherited diseases in which defective genes are known and have been cloned. In some cases, the function of these cloned genes is known. In general, the above disease states fall into two classes: deficiency states, usually of enzymes, which are generally inherited in a recessive manner, and unbalanced states, at least sometimes involving regulatory or structural proteins, which are inherited in a dominant manner. For deficiency state diseases, gene transfer could be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. Thus the methods of the present invention permit the treatment of genetic diseases, e.g., to screen compounds for activity useful in combatting the disease state. As used herein, a disease state is treated by partially or wholly remedying the deficiency or imbalance which causes the disease or makes it more severe. The use of site-specific integration of nucleic sequences to cause mutations or to correct defects is also possible.

In one particularly preferred embodiment, the present invention is employed to express an exogenous CFTR protein in respiratory epithelium. According to this embodiment, it is preferred to use an AdV transfer vector carrying the CFTR gene. The AdV-CFTR is directly linked to the 4-amino substituent of the dinucleotide UP₄C with a sulfo-NHS activated agent, as described above. Binding of UP₄C to the P2Y₂ receptor on the apical surface of the respiratory epithelium will induce internalization of the entire UP₄C-AdV-CFTR complex into epithelial cells.

I. Pharmaceutical Formulations, Subjects, and Methods of Administration

Suitable subjects to treated according to the present invention include both avian and mammalian subjects, preferably mammalian. Any mammalian subject in need of being treated according to the present invention is suitable. Human subjects are preferred. Human subjects of both genders and at any stage of development (i.e., neonate, infant, juvenile, adolescent, adult) can be treated according to the present invention. Human subjects afflicted with cystic fibrosis are preferred.

The compounds of the invention may be present in the form of theirs pharmaceutically acceptable salts, such as, but not limited to, an alkali metal salt such as sodium or potassium; an alkaline earth metal salt such as manganese, magnesium, or calcium; or an ammonium or tetraalkyl ammonium salt, i.e., NX4⁺(wherein X is C₁₋₄). Pharmaceutically acceptable salts are salts that retain the desired biological activity of the parent compound and do not impart undesired toxicological effects.

Active compounds of the present invention can be administered to a subject in need thereof by any suitable means including oral, rectal, transmucosal, topical or intestinal administration; parenteral delivery, including intramuscular, subcutaneous, intramedullary injections, as well as intrathecal, direct intraventricular, intravenous, intraperitoneal, intranasal, or intraocular injections. Alternately, one may administer the compound in a local rather than systemic manner, for example, in a depot or sustained release formulation. Administration to the lungs is preferred.

Active compounds disclosed herein may be administered to the lungs of a subject by any suitable means, but are preferably administered by administering an aerosol suspension of respirable particles comprised of the active compound, which the subject inhales. The respirable particles may be liquid or solid.

Aerosols of liquid particles comprising the active compound may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic with body fluids by the addition of, for example, sodium chloride.

Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100% w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 150 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

The aerosol, whether formed from solid or liquid particles, may be produced by the aerosol generator at a rate of from about 10 to 150 liters per minute, more preferably from about 30 to 150 liters per minute, and most preferably about 60 liters per minute. Aerosols containing greater amounts of medicament may be administered more rapidly.

The dosage of the active compounds disclosed herein or pharmaceutically acceptable salt thereof, will vary depending on the condition being treated and the state of the subject, but generally may be an amount-sufficient to achieve dissolved concentrations of active compound on the airway surfaces of the subject of from about 10⁻⁹ or 10⁻⁷ to about 10⁻³ Moles/liter, and more preferably from about 10⁻⁶ to about 3×10⁻⁴ Moles/liter. Depending upon the solubility of the particular formulation of active compound administered, the daily dose may be divided among one or several unit dose administrations. Other compounds may be administered concurrently with the active compounds, or salts thereof, of the present invention.

Solid or liquid particulate pharmaceutical formulations containing active agents of the present invention should include particles of respirable size: that is, particles of a size sufficiently small to pass through the mouth and larynx upon inhalation and into the bronchi and alveoli of the lungs. In general, particles ranging from about 1 to 5 microns in size (more particularly, less than about 4.7 microns in size) are respirable. Particles of non-respirable size which are included in the aerosol tend to be deposited in the throat and swallowed, and the quantity of non-respirable particles in the aerosol is preferably minimized. For nasal administration, a particle size in the range of 10-500 μm is preferred to ensure retention in the nasal cavity.

In administering the active compounds of the present invention, they may be administered separately (either concurrently or sequentially) or, alternatively and preferably, they may be pre-mixed and administered as preformed conjugates. As an illustrative example, as suitable dose of a transfer vector carrying a heterologous nucleic acid of interest, can be pre-mixed with a targeting molecule (i.e., a bispecific bridging antibody, a peptide, biotin-UTP, etc.) and the complex administered to the subject.

In the manufacture of a formulation according to the invention, active agents or the physiologically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a capsule, which may contain from 0.5% to 99% by weight of the active compound. One or more active compounds may be incorporated in the formulations of the invention, which formulations may be prepared by any of the well-known techniques of pharmacy consisting essentially of admixing the components.

Compositions containing respirable dry particles of active compound may be prepared by grinding the active compound with a mortar and pestle, and then passing the micronized composition through a 400 mesh screen to break up or separate out large agglomerates.

The pharmaceutical composition may optionally contain a dispersant which serves to facilitate the formation of an aerosol. A suitable dispersant is lactose, which may be blended with the active agents in any suitable ratio (e.g., a 1 to 1 ratio by weight).

In summary, the transfer vectors of the present invention can be used to stably transfect either dividing or non-dividing cells, and stably express a heterologous gene. Using this vector system, it is now possible to introduce into dividing or non-dividing cells, genes which encode proteins that can affect the physiology of the cells. The vectors of the present invention can thus be useful in gene therapy for disease states, or for experimental modification of cell physiology, such as to produce models useful in screening compounds for particular physiologic activity.

Having now described the invention, the same will be illustrated with reference to certain examples which are included herein for illustration purposes only, and which are not intended to be limiting of the invention.

### EXAMPLE 1

### Models of Human Airway Epithelium

Epithelial cells are derived from CF and non-CF nasal and bronchial airway epithelia using procedures similar to those described by Gray et al. 1996. Am. J Respir. Cell Mol. Biol. 14, 104-112. Resected nasal turbinates or portions of mainstem/lobar bronchi representing excess donor tissue are obtained at the time of lung transplantation under the auspices of the University of North Carolina at Chapel Hill Institutional Committee on the Protection of the Rights of Human Subjects. Epithelial cells are removed from the specimens by protease XIV digestion as described (Wu, R., et al., 1985. Am. Rev. Respir. Dis. 132, 311-320), but omitting the filtration step. 1-2×10⁶ cells are plated per 100 mm tissue culture dish in modified LHC9 medium. Lechner, J. F. and Laveck, M. A. 1985. J. Tiss. Cult. Meth. 9, 43-48. The modifications include increasing the EGF concentration to 25 ng/ml, adjusting the retinoic acid concentration to 5×10⁻⁸ M, and supplementation with 0.5 mg/ml bovine serum albumin and 0.8% bovine pituitary extract. At approximately 75% confluence, the cells are harvested by trypsinization and passage 1 cells are plated at a density of 2.5×10⁵ cells on Transwell-Col inserts (Coming-Costar, 24 mm diameter, 0.4 μm pore size), in modified medium. The medium is similar to the supplemented LHC9 except that a 50:50 mixture of LHC Basal (Biofluids) and DMEM-H is used as the base, amphotericin and gentamycin are omitted and the EGF concentration is reduced to 0.5 ng/ml. After the cells grow to confluence (4-6 days) the apical surface of the cultures are given an air-liquid interface for another 25-30 days until use.

Initial histological analyses of human well differentiated (WD) cultures derived from non-CF nasal airways after 34 days of culture indicate that the epithelium is pseudostratified mucociliary, with abundant cilia and cell-types representative of those present in human nasal airways in vivo.

### EXAMPLE 2

### Seven Transmembrane Receptor Expression on the Apical Membrane of WD Cells

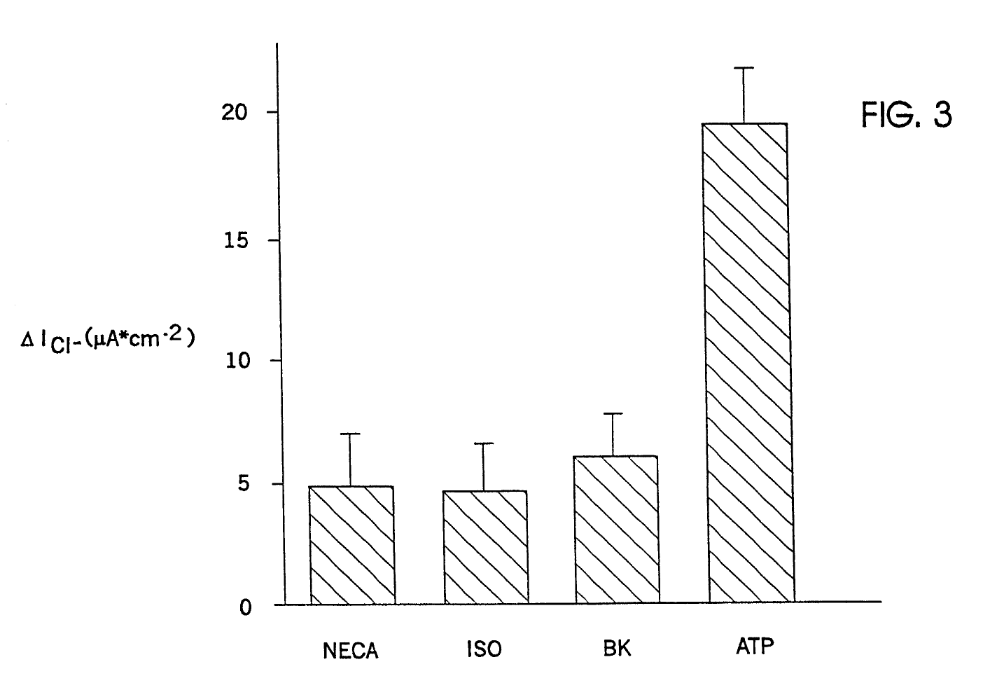

Investigations were carried out to identify which 7-TM receptors, if any, are localized in the apical membrane of WD human airway epithelial cells. Cultures of WD cells were exposed to NECA (an A_{2b} receptor agonist), isoproterenol, bradykinin, or ATP (all at 10⁻⁴M), and Cl-secretory responsiveness was determined. As shown in FIG. 3, a Cl-secretory response was detected in the presence all of the agonists, however, the greatest response was observed in the presence of ATP. These results strongly suggest that functional adenosine, β-adrenergic, bradykinin and purino receptors are present on the apical surface of airway epithelia.

### EXAMPLE 3

### Binding and Internalization of AdV in Human PD and WD Airway Epithelial Cells

Experiments showing that adenovirus vector (AdV)-internalization, not AdV-binding, is the rate-limiting step resulting in low efficiency gene transfer to RTE WD cultures are repeated with human cultures to determine if the same rate-limiting step is present. If this is indeed the case, then although the cells have a reduced rate of internalization, it may be possible to increase gene transfer efficiency to WD cultures by enhancing the amount of AdV that binds to these culture-types. For a given concentration of AdV, exposed to either poorly differentiated (PD) or well differentiated (WD) cultures, only approximately 0.1-1% of the total AdV exposed to cells remains attached after washing. Enhancement of AdV-binding above that achieved with a single exposure leads to an increase in gene transfer, since increasing the binding of AdV to cells will increase the probability that an internalization event leads to AdV entry.

To determine the rate-limiting step for inefficient gene transfer in human cultures, PD and WD cultures are exposed to ³⁵S-AdSVLacZ (1.2×10¹⁰ p) for analyses of AdV-binding, internalization and transgene expression in the human cultures. To investigate the effect of increasing the concentration of AdV and/or the duration of exposure to AdV, PD and WD cultures are exposed to ³⁵S-Ad5VLacZ (Pickles, R. J., et al., 1996. Human Gene Therapy 7, 921-931) at a range of concentrations (10⁷-10¹² p/ml) for a number of time points (1-24 hrs) at 4° C., after which cultures are washed in medium and then divided into three groups for analyses. Binding is measured as cell-associated radioactivity. Internalization of bound AdV is measured by transferring the cultures to 37° C. for 6 hrs followed by measurement of cell-associated radioactivity after removal of non-internalized radioactivity. Expression is measured by transferring the cultures to 37° C. for 48 hrs before measuring β-gal activity. Radioactive counts per minute (CPM) and β-gal activity are standardized with respect to the nominal surface area of the culture surface because the apical surface area of cells exposed to vector is the most appropriate denominator, as it allows direct comparison to the epithelium in vivo.

It is likely that with PD cells, for a specific incubation time, a 10-fold increase in concentration will result in a 10-fold increase in AdV attachment, internalization and gene expression as long as saturation of receptor uptake and expression systems does not occur. With WD cultures, although a 10-fold increase in AdV attachment is expected, the corresponding 10-fold increases in internalization and expression are not. These data indicate that increased binding alone does not overcome the rate-limiting step (internalization) into WD cultures.

### EXAMPLE 4

### Targeting of AdV Vectors to the P2Y₂ Receptor

To test the concept that the P2Y₂ receptor is a candidate receptor for targeting based on the ability to bind and internalize an exogenous ligand, we have obtained CHO and A9 cells (both of which are not transducible by AdV) that express the HA-tagged human P2Y₂ receptor (HA tag on the extracellular N-terminus) by retroviral gene transfer. HA-P2Y₂ receptor expressing A9 cells, but not control cells, stain with fluorescently labeled anti-HA Abs under resting conditions. With agonist [ATPγS (10⁻⁴ M)] exposure, approximately 80% of the receptors are internalized within 45 minutes. The internalization of P2Y₂ receptor is mediated via coated pits.

Next, a bi-specific antibody (bs-Ab) approach was used to test whether P2Y₂ receptor could mediate gene transfer. Antibody HA.11 (BabCO) against influenza hemagglutin (anti-HA) is directed against the HA-epitope inserted into an extracellular domain of the human P2Y₂-receptor which is expressed in 1321N1 human astrocytoma cells. The bridging antibody is produced by reacting an anti-fiber (knob) antibody with m-Maleimidobenzoyl-N-hydroxysulfosuccinimide ester (Sulfo-MBS, Pierce, Rockford, Ill.) or N-(γ-maleimidobutyryloxy) sulfosuccinimide ester (Sulfo-GMBS, Pierce, Rockford, Ill.) at neutral pH. After reduction of anti-HA by mercaptoethylamine, and desalting, the two antibodies are mixed, enabling disulfide cross-link formation. Bi-functional antibody is purified by sequential chromatography over fiber protein and HA columns.

Using this bs-Abs against the Ad fiber (knob) and the HA epitope tag (αfiber×αHA), binding of bs-Ab and AdV to HA-P2Y₂ receptor, but not to null expressing A9 cells has been demonstrated. AdV bound to A9-HA-P2Y₂ cells in the presence, but not the absence, of bs-Ab.

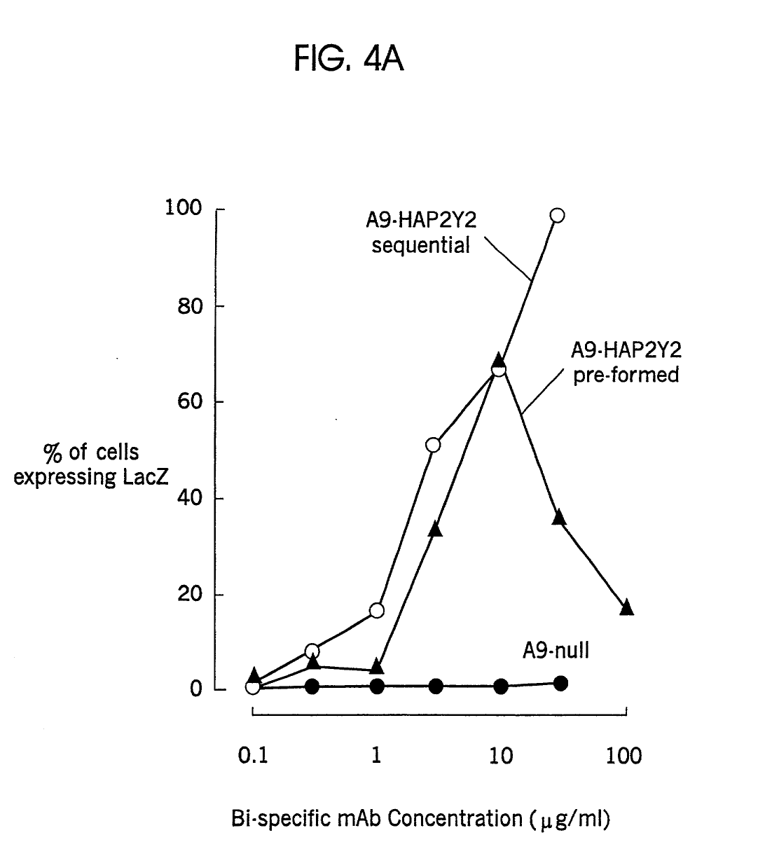

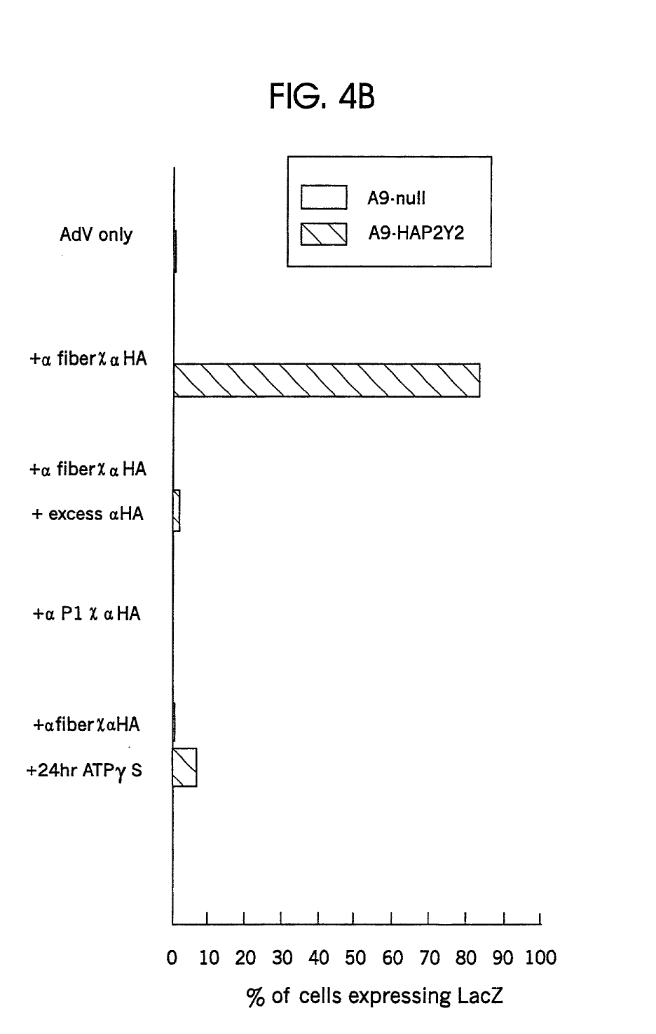

More importantly, P2Y₂ receptor specific gene transfer in A9 and CHO cells has been achieved using the bs-Ab approach. In one protocol employing: (1) sequential exposure at 4° C. of HA-P2Y₂ receptor expressing A9 versus null vector-expressing A9 cells to varying concentrations of bs-Abs (anti-HA/anti-fiber knob; produced by Dr. R. Pickles in the laboratory of Dr. D. Segal at the NIH) followed by AdV-IacZ (particles per cell=10⁴) (Pickles, R. J., et al., 1996. Human Gene Therapy 7, 921-931); (2) incubation for 1 hour at 37° C. with agonist (ATPγS, 10⁻⁴ M), followed by incubation for 24 hour in medium; and (3) quantitation of gene transfer efficiency by counting and calculating the percent lacZ positive cells, it was observed that HA-P2Y₂ receptor expressing A9 cells are transduced by Ad-lacZ as a function of the concentration of bs-Ab, whereas null A9 cells are not (FIG. 4A). Indeed, it appears that nearly 100% gene transfer efficiency is approached with 30 μg/ml of bs-Abs.

In a second protocol using preformed conjugates, augmentation of gene transfer that peaked at ˜10 μg/ml bs-Ab was observed (FIG. 4A). The fall in transduction efficiency at higher bs-Ab concentrations may reflect competition by unbound bs-Ab for the target.

The specificity of the gene transfer observed with bs-Ab in A9-HA-P2Y₂-R cells was evaluated (FIG. 4B). HA-P2Y₂-A9 cells exposed to Ad-LacZ and ATPγS showed almost 100% expression of LacZ. In contrast, almost no LacZ expression was observed with an irrelevant bs-Ab (anti-Ha/anti-AD P1 epitope), nor in the presence of a 40× excess of free anti-HA antibody, nor in cells that had been pre-exposed for 24 hour to a high concentration of agonist (thereby inducing cell-surface receptor down-regulation). These data establish that the HA-P2Y₂ receptor mediates gene transfer via specific interactions with anti-HA bs-Abs. Finally, Similar increases in gene transfer to HA-P2Y₂-R A9 cells with bs-Abs against HA and biotin (αHA/αbiotin) and biotinylated adenoviral vectors have been demonstrated.

### EXAMPLE 5

### Exploitation of Other Cellular Uptake Mechanisms to Increase AdV-Entry into WD Cultures

The effectiveness of gene transfer by targeting AdV to receptors that undergo internalization is dependent on the internalization efficiency of the receptor. To test the internalization efficiency of P2Y₂-receptors, a human P2Y₂-receptor (with an influenza hemagluttin (HA) epitope-tag inserted into the extracellular domain) has been overexpressed in 1321N1 human astrocytoma cells. (S. Sromek and T. K. Harden, Molecular Pharmacology 54, in press (1988)). P2Y₂-HA expressing cells were incubated at 37° C. with anon-hydrolyzable ATP analogue, ATPγS. At specific time points, after agonist addition, the cells were fixed without permeabilization in 4% paraformaldehyde and washed. Monoclonal anti-HA antibody was incubated with the cells followed by incubation with Cy3-conjugated goat anti-mouse IgG secondary antibody (Jackson Immuno Research Labs). The availability of P2Y₂-HA receptors to the anti-HA was visualized with a fluorescent microscope. The astrocytoma cells were fixed and immunostained for the presence of P2Y₂-HA-receptor in the absence of ATPγS and after 30 min ATPγS exposure. In the continued presence of agonist there is a clear loss of immunoreactivity from the plasma membrane and the punctate fluorescent signals, indicating sequestration into endosomes. With ELISA assays internalization was quantitated to occur with an efficiency of 80% (loss of receptor sites with ATPγS exposures of 1 hr, Dr. Ken Harden, personal communication). This indicates that in the astrocytoma cell-line, internalization of activated receptors occurs efficiently.

### EXAMPLE 6

### Gene Transfer Across the Apical Membrane of Polarized Epithelium

Confluent MDCK renal cells were used as a model of a polarized, AdV-resistant epithelium. In MDCK cells expressing the HA-P2Y₂ receptor, the receptors were localized on the apical surfaces. Cells were exposed to anti-HA and anti-mouse IgG FITC at 4° C., and receptor localization determined by confocal microscopy. It was observed that HA-P2Y₂ receptors in the apical membrane of polarized MDCK cells desensitizes, in part, by internalization of receptors. Bs-Abs directed to HA-P2Y₂ receptors sequentially administered with AdV-GFP (green fluorescent protein) and ATPγS transduce HA-P2Y₂ receptor, as compared with Neo-expressing MDCK cells. Thus HA-P2Y₂ receptor specific gene transfer has been achieved in polarized, AdV-resistant cells.

### EXAMPLE 7

### Gene Transfer in A9 Cells Expressing the Bradykinin II (B2K) Receptor and in CHO Cells Expressing the P2Y₂ or β₂ Receptors

The general applicability of the above approach has been demonstrated in other cell types and with other receptors.

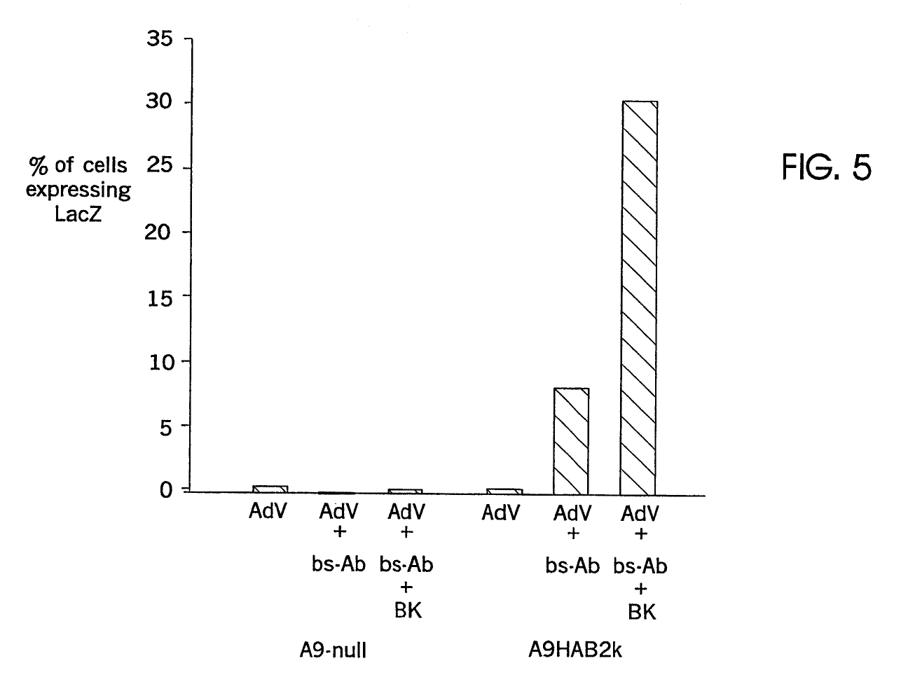

A9 cells expressing the HA-epitope tagged BKII receptor and A9 cells expressing neomycin alone were exposed to bi-specific antibodies (anti-fibre-knob×anti-HA) and AdV in the absence and presence of bradykinin. Briefly, cells at 4° C. were incubated in the absence or presence of bi-specific antibody (bs-Ab, 10 μg/ml for 2 hrs), washed and exposed to AdVLacZ (10¹⁰ particles for 2 hrs), washed and exposed to bradykinin (BK, 1 μM for 2 hrs at 37° C.). The cells were then maintained at 37° C. for 24 hrs until gene expression was assessed by standard techniques. FIG. 5 shows that efficient gene expression occurs only in HAB2k-expessing cells incubated with both bs-Ab and AdV with enhancement of gene transfer by activating the receptor with agonist.

Almost no expression was observed with AdV alone, and only modest levels were observed in the presence of AdV+bs-Ab (indicating low-level receptor tum-over even in the absence of ligand). Only negligible LacZ expression was observed in null A9 cells regardless of treatment.

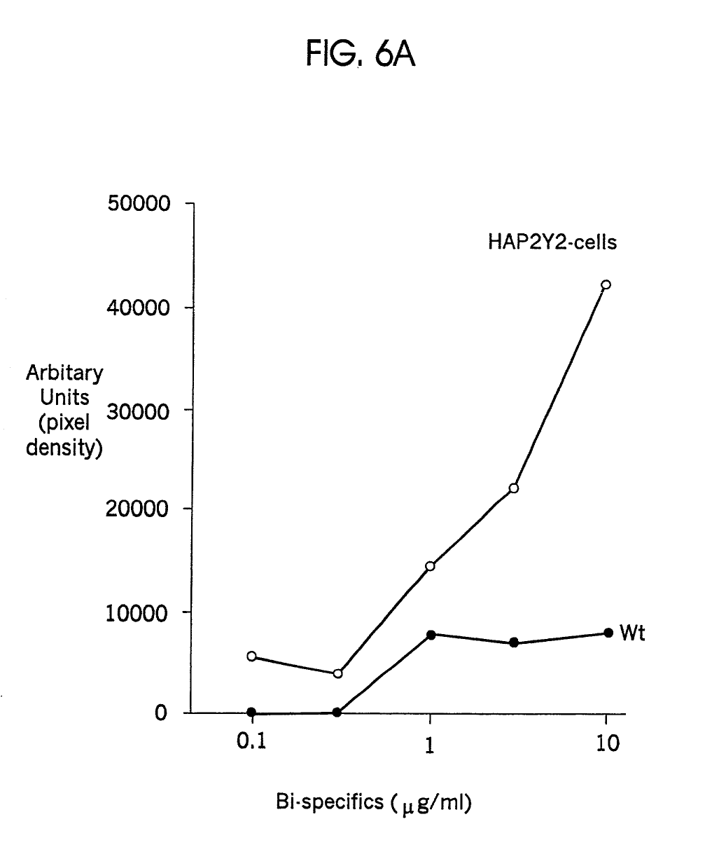

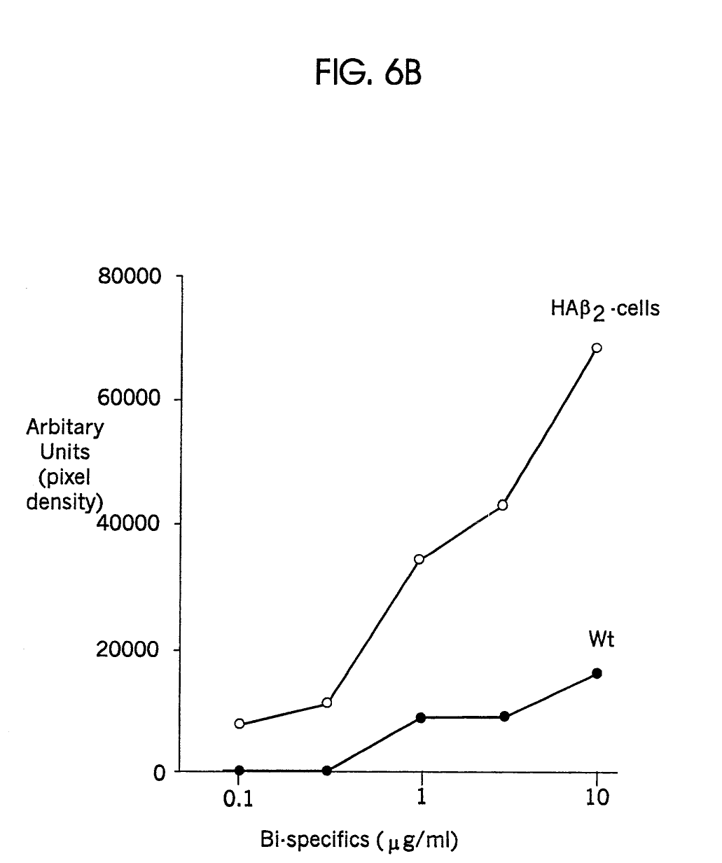

In additional studies, CHO cells (Chinese Hamster Ovary cells which lack the AdV attachment receptor) expressing the HA-epitope tagged 2-adrenoreceptor and wild-type (Wt) CHO cells were sequentially exposed to increasing concentrations of bi-specific antibodies (anti-fibre-knob×anti-HA) and AdV and finally to isoproterenol. Briefly, cells at 4° C. were incubated in the absence or presence of bi-specific antibody (bs-Ab, 0.1-10 μg/ml for 2 hrs), washed and exposed to AdVLacZ (10¹⁰ particles for 2 hrs), washed and exposed to isoproterenol (10 μM for 2 hrs at 37° C.). The cells are then maintained at 37° C. for 24 hrs until gene expression was assessed by densitometry of LacZ expressing cells shown as arbitrary units. FIG. 6A-B shows bs-Ab dose-dependent increases in gene expression only in CHO cells expressing HA-P2Y₂-R (FIG. 6A) or HA-β₂ (FIG. 6B) receptors.

### EXAMPLE 8

### Biotinylated Agonists

Another approach to target vectors to the P2Y₂ receptor (or any other 7-TM receptor) is by chemical linkage to a modified agonist/antagonist molecule as a target molecule. A prototype agonist-linker is biotin (B)-UTP, which contains a 16 atom linker connecting the 5 position of the pyrimidine base to biotin. B-UTP is an agonist of P2Y₂ receptors (FIG. 7). Fluorescence studies have demonstrated that when CHO cells expressing P2Y₂ receptors are exposed to B-UTP conjugated to streptavidin-Texas Red (TR), the B-UTP triggers and is internalized with the P2Y₂ receptors.

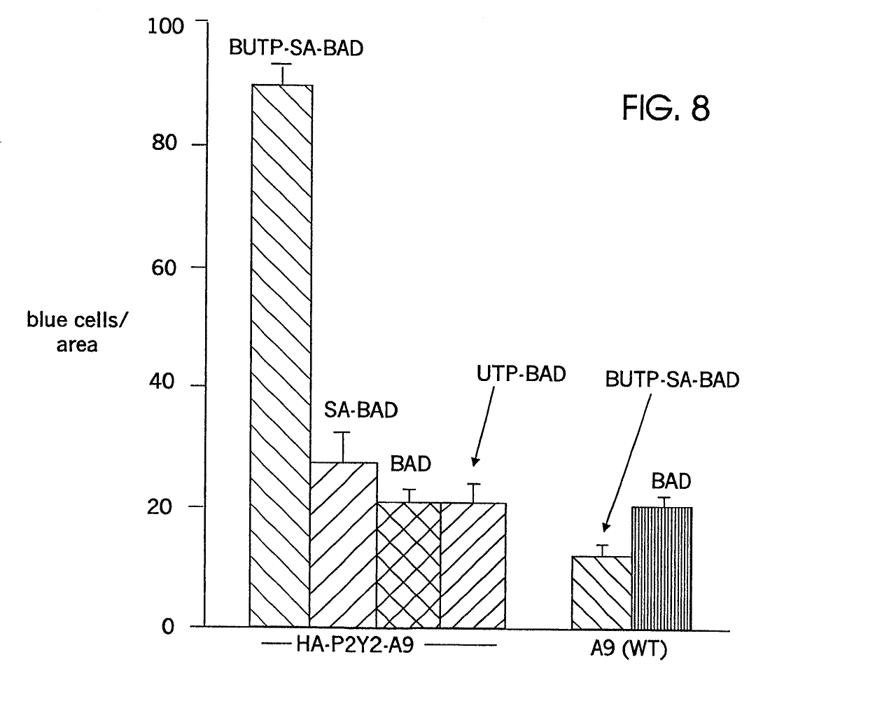

In a further study, gene targeting by B-UTP was evaluated in A9 cells expressing HA-P2Y₂ receptors. HA-P2Y₂-A9 cells were sequentially exposed to B-UTP, streptavidin (SA), and B-AD (biotin labeled adenovirus expressing LacZ). Approximately, 90% of HA-P2Y₂-A9 cells demonstrated LacZ expression (FIG. 8). In contrast, only low levels of LacZ expression were observed in wild-type A9 cells.

### EXAMPLE 9

### Dinucleotide Agonists

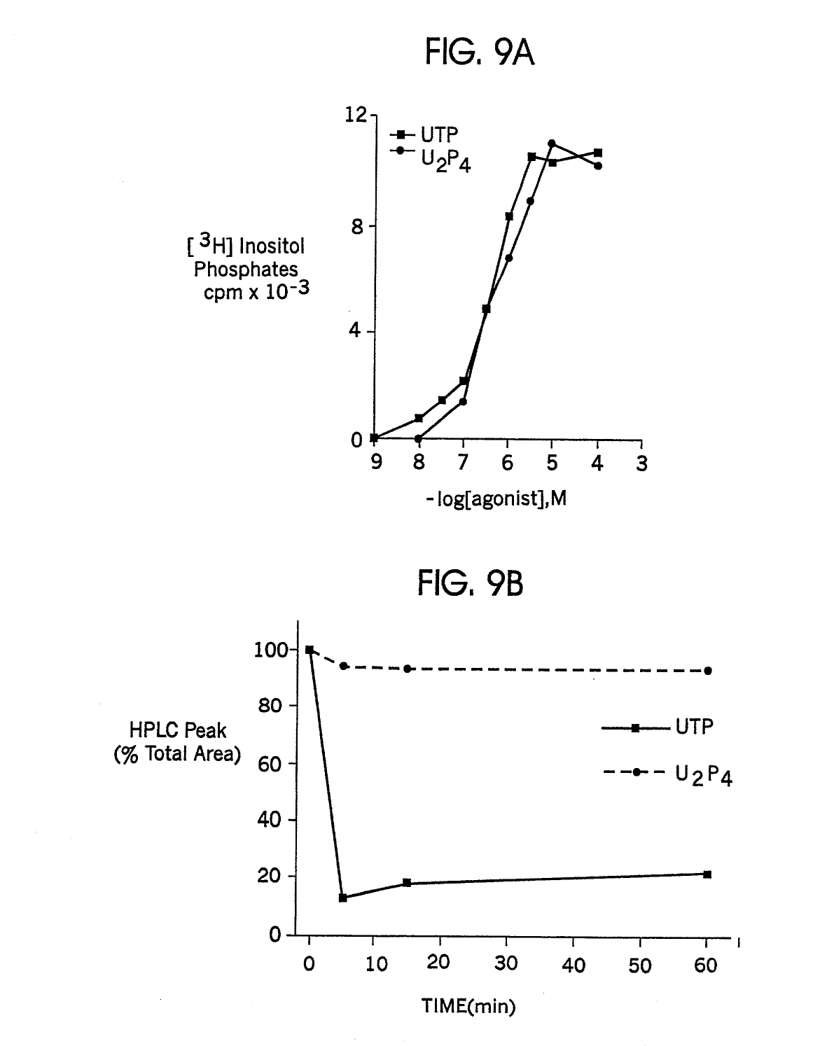

Biologically active analogs of UTP that are more resistant to biological hydrolysis than UTP have been developed for linkage to vectors. The dinucleotide U₂P₄ has useful properties. As shown in FIG. 9, U₂P₄ is equipotent with UTP in inducing a biological response (inositol phosphate release) and is resistant to hydrolysis in cystic fibrosis (CF) sputum.

### EXAMPLE 10

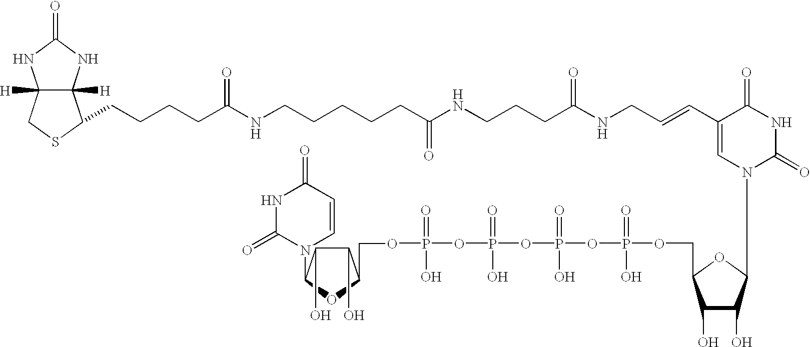

Uridine 5′-monophosphate, free acid (1 mmol) (Aldrich, St. Louis, Mo.) was dissolved in dimethylformamide (DMF, 0.5 mL) and tributylamine (0.5 mL) (Aldrich) and evaporated under reduced pressure to form an oil. The oil was redissolved in dry DMF (0.5 mL) and concentrated again. This evaporation procedure was repeated one more time and the final oil was dissolved in 2.5 mL dry DMF to which was added carbonyldiimidazole (2 mmol) (Aldrich). After stirring at 37° C. for 2 hours this stock solution of activated UMP was set aside. 20 vials of Bio-16-UTP (250 umol each) (Boeringer Mannheim) were transferred with 100 μL of water for each vial into one larger vial. The pooled solution was passed through a column (0.5×3 cm) of Dowex 50H⁺resin (Dow Chemical Co.) and flushed with three column volumes of water. To the eluate was added DMF (2 mL) and tributylamine (0.25 mL) and the solution was evaporated to an oil under reduced pressure. The oil was dissolved in dry DMF (0.5 mL) and the evaporation repeated two times. The final oil was dissolved in dry DMF (0.5 mL) and tributylamine (40 μL). To this solution was added 200 μL of activated UMP solution prepared above. The reaction mixture was allowed to stir at 37° C. for 18 hours and then more activated UMP solution (100 μL) was added and the temperature raised to 50° C. After two hours at 50° C. the reaction mixture was cooled to room temperature and water (0.5 mL) was added. The suspension was filtered through a nylon filter and the solution was concentrated and transferred to an HPLC vial insert. The crude reaction mixture was purified by preparative HPLC (AX300 column; 1M NH₄HCO₃ water/buffer gradient; 4 mL/min) to yield 1.2 mg of Bio-16-UP₄U containing 50% unreacted starting material (Bio-16-UTP).

Example 11

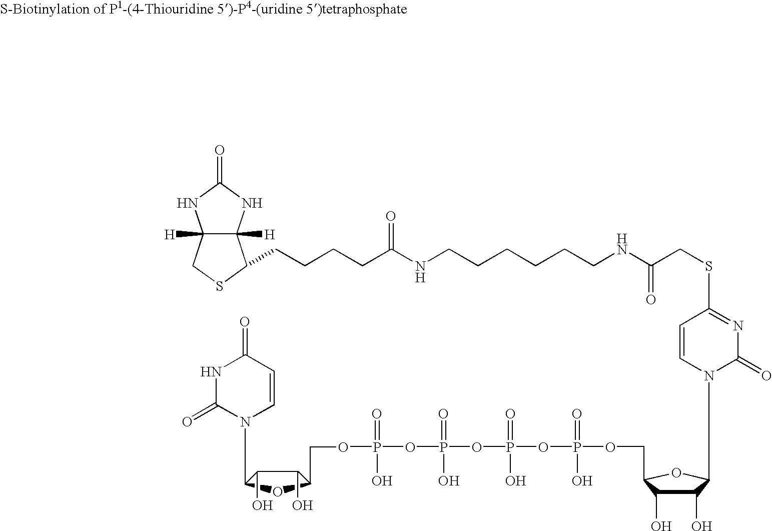

A solution of the sodium salt of P¹-(uridine 5′)-P⁴-(4-thiouridine 5′)tetraphosphate (4.825 μmol) in water (1.0 mL) was stirred with N-iodoacetyl-N′-biotinylhiexylenediamine (2.46 mg, 4.825 μmol) at room temperature for 48 h. The solution was filtered and subjected to ion exchange HPLC (PerSeptive Biosystems, POROS HQ/H column, 4.6×100 mm, gradient 0-0.66 M ammonium bicarbonate over 20 min. 5.0 mL/mm) in ten aliquots. The main fractions from each run (4.7-5.4 min) were combined and lyophilized to yield P¹(4(N′-biotinylaminohexylcarbamoylmethylthiouridine 5′)-P⁴-(uridine 5′)tetraphosphate (1.426 mmol, 29.6% yield, quantitated approximately by comparison of its absorbance at λₘₐₓ 305 nm with that of a standard solution of 4-methylthiouridine monophosphate). ³¹P NMR (D₂O, H₃PO₄′std.) δ (ppm) −22.55 (m, 2P), −10.96 (m, 2P). ¹H NMR (D2O, TMS std.) 1.20-1.60 (m, 14H); 1.74 (s, 2H); 2.08 (t, J=6.9 Hz, 2H); 2.59 (d, J=12.9 Hz, 1H); 2.81 (dd, J=13.0, 5.0 Hz, 1H); 2.97 (m, 2H); 3.03 (m, 3H); 3.13 (m, 1H); 4.044.28 (m, 14H); 4.42 (m, 1H); 4.69 (m, partially obscured by H₂O, 10H); 5.78, (m, 4H); 6.61 (d, J=6.6 Hz, 1H); 7.75 (d, J=8 Hz, 1H); 8.08 (d, J=7.1 Hz, 1H).

### EXAMPLE 12

### Investigation into the AdV-Internalization Processes in Human PD and WI Cultures

The α_{V}β_{3/5} integrins are reported to mediate internalization but not attachment of Ad into epithelial cells in vitro. The localization of the membrane-bound (α_{V}β_{3/5} integrins to the apical and/or basolateral membranes of WD cultures is crucial in understanding the roles of these molecules in AdV-mediated gene transfer. The availability of a number of different antibodies to these integrins allows their location in polarized epithelia to be determined. These integrins are also receptors for peptides containing RGD amnino acid sequences. A number of studies have shown that RGD-peptides inhibit AdV-mediated gene transfer to epithelial cells by interaction with the α_{V}β_{3/5} integrins. This Example illustrates the effects of RGD peptides on AdV-binding, internalization and transgene expression in PD and WD cultures.

An integrin antibody-specific immnunoprecipitation procedure has been developed initially with rat trachial epithelium (RTE) well differentiated (WD) cultures to localize the α_{V}β_{3/5} integrins. Since antibodies to these integrins are not commercially available, we have obtained an antibody (R838, a kind gift from Dr. Steven Albelda, Univ. of Penn, PA), raised against the human endothelial cell vitronectin receptor which has been found to cross-react with rat α_{V}β_{3/5} integrins³⁷ is used. Briefly, either the apical or basolateral domains of WD cultures were exposed to Sulfo-NHS-Biotin (0.5 mg/ml, Pierce) at 4° C. to biotinylate only external membrane proteins. After solubilization of the cells in a non-denaturing lysis buffer (in the presence of protease inhibitors), proteins were immunoprecipitated and separated by Western analysis on a 4-12% acrylamide gel (Novex) under non-reducing conditions. The biotinylated proteins were probed with streptavidin-conjugated peroxidase secondary antibody and detected by ECL analysis (Supersignal CL-HRP, Pierce). The biotinylated proteins identified by R838 are shown in FIG. 5 (bands at 125 kD correspond to the α_{V} and 97 kD are β_{3/5} subunits) and appear to be present in the basolateral membranes of RTE cells and in HeLa cells but absent from the apical membrane of the WD cultures, suggesting that rat vitronectin receptors may not be located apically in these culture-types. The absence of these integrins in the apical membrane could account for the low rate of AdV-internalization into these cultures.

### EXAMPLE 13

### Identification and Localization of Integrins Present on Human PD and WD Cultures

Proteins from the apical and/or basolateral membranes of human PD and WD cultures are selectively isolated by exposing individual surfaces to Sulpho-NHS-Biotin at 4° C. Standard immunoprecipitations experiments are performed with selective human antibodies (LM609, α_{V}β₃; P1F6, α_{V}β₅; VNR147, α_{V}; P4G11, β₁; CD61, β₃; anti-β₅ and R838, α_{V}β_{3/5}; all obtained from Chemicon Inc, CA.). This procedure allows for detection and localization of the α_{V}β integrins to the apical and/or basolateral membrane.

### EXAMPLE 14

### Interference of AdV-Internalization by RGD Peptides.

Adenoviral attachment, internalization and transgene expression in PD and WD cultures is measured, as described above, in the absence and presence of RGD-peptides. Hexa-peptides, the bioactive GRGDSP (Gibco-BRL) and the inactive control peptide GRGESP (Gibco-BRL) are administered to PD and WD cultures at a final concentration of 0.1-4.0 mg/ml for 2 hrs at 4° C. before the addition of AdV (10¹⁰ p/ml). Cyclical RGD peptides (Immunodynamics, La Jolla, Calif.), reported to be more potent at reducing AdV-mediated gene transfer, are also used. Analyses of AdV-attachment, internalization and transgene expression are performed as described above.

### EXAMPLE 15

### Investigation of the Cellular Uptake Processes for AdV-Entry

Initial attachment of AdV to epithelial cells occurs via the fiber (knob) protein. It is unclear whether fiber protein alone is sufficient to trigger internalization and endosome formation or whether the role of fiber is to aid the virus to locate and exploit an inherent endocytotic event. Internalization of AdV into the cytoplasm however, is mediated, in part, by α_{V}β_{3/5} integrins. T. J. Wickham et al., Cell 73, 309-319 (1993). It has been speculated that α_{V}β_{3/5} integrins are absent or low in number in the apical membranes of both WD cultures and cartilaginous airway epithelium (M. J. Goldman et al, J. Virol. 69, 5951-5958 (1995)), possibly resulting in both the low rate of internalization and gene transfer efficiency in these cell-types. Therefore, the potential cellular uptake processes that may be responsible for entry of AdV into cells are investigated. First, to understand the functional role of the fiber (knob) protein-cell interaction, knob protein is conjugated to fluorescent microspheres of the same diameter as Ad and the initial interactions of the knob-spheres on PD and WD cells are assessed by confocal microscopy. Increased specific or non-specific binding may increase internalization and possibly expression. Second, human α_{V}β₅ integrin is overexpressed in WD cultures to direct this protein to the apical membrane. This is used to test the hypothesis that α_{V}β₅ expression on the apical membrane is the rate-limiting step for internalization and hence gene expression. Third, in order to understand the cell-entry pathways utilized by Ad, cell-lines that are either deficient or competent at coated pit receptor-mediated endocytosis are tested. The hypothesis that high concentrations of AdV may use non-specific entry pathways to gain access into cells is tested. Finally, a strategy is tested to increase the internalization efficiency of AdV into WD cells by exploitation of other cellular uptake mechanisms, i.e., targeting AdV to specific receptor types that undergo endocytosis when stimulated by exogenous ligands.

### EXAMPLE 16

### AdV Internalization into HeLa Cell-Lines with Competent and Defective Receptor-Mediated Endocytosis

Adenoviral entry into cells may reflect uptake by a number of cellular pathways i.e., receptor-mediated endocytosis via coated pits, non-specific pinocytosis, or phagocytosis. (R. M. Steinman et al., J. Cell Biol. 96, 1-27.) The role of coated-pit receptor-mediated endocytosis and non-specific pinocytosis on AdV-entry into cell-lines which have either competent or defective receptor-mediated endocytosis is studied. HeLa cell mutants have been produced which can overexpress either wild-type dynamin protein or a mutant form, mDyn (controlled by a TET-inducible promoter). (H. Damke et al., J. Cell Biol. 127, 915-934.) Normally, dynamin is responsible for coated pit endosome formation and functions by ‘pinching’ off invaginations in the plasma membrane. HeLa cells overexpressing wild-type dynamin show no functional or morphological alteration of uptake processes compared to parent cells. Cells overexpressing nmDyn form coated pits and invaginate the plasma membrane but fail to bud coated vesicles into the cytoplasm. Ligands for receptor-mediated endocytosis (EGF and transferrin) fail to be internalized into cells expressing niDyn, but ligand-receptor binding, coated pit assembly, recruitment of receptors into coated pits and invagination of the plasma membrane are all unaffected. In the absence of receptor-mediated endocytosis, non-specific pinocytosis initially remains unaltered but with time is upregulated to compensate for the loss of receptor-mediated endocytosis.

### EXAMPLE 17

### Uptake Processes for AdV-Entry into HeLa Cells

HeLa cells either overexpressing wild-type or mDyn (gift of Sandra Schmid, Scripps Research Institute, La Jolla, CA) are used to study the uptake processes that are prevalent for AdV-entry into these cells. A range of AdV concentrations are studied to determine if high titre AdV leads to cell-uptake by non-specific processes. Briefly, monolayers of mutant HeLa cells, grown on plastic, expressing wild-type or mdyn dynamin will be exposed to Ad5VLacZ (10⁶-10¹¹ i.u./ml, corresponding to an MOI˜1-10⁵) at 4° C. for 2 hrs and then transferred, without washing, to 37° C. for time-periods of 0-24 hr, at which point the cells will be washed and maintained at 37° C. for 48 hrs before β-gal enzymatic assays are performed. Differences in the gene expression observed in the two cell-lines at specific time-points will reflect the participation of receptor-mediated endocytosis on AdV-entry. In conjunction, comparative studies with fluorescent microspheres (with and without attached knob-protein) to delineate the interaction of these proteins with specific and non-specific uptake processes are also conducted.

### EXAMPLE 18

### Analysis of Transgene Expression in Human PD and WD Cultures

PD and WD cultures are exposed to Ad5VLacZ by application to either the apical and/or basolateral membranes over a range of viral titres (10⁶-10¹¹ infectious units/ml: corresponding to MOI range of 1-10⁵) with incremental exposure times (1-24 hrs), to study the effects of concentration and time on the gene expression obtained. Vectors used in this study are produced and titred by the UNC Gene Therapy Core. Incubations are performed at 37° C. and/or 4° C. The former temperature allows potential cellular uptake processes to be studied, while the latter temperature, is a standardized technique for measuring the initial attachment of ligands to their receptors, in the absence of receptor recycling and/or internalization. Gene expression is assessed 48 hrs after initial exposure to AdV by both qualitative and quantitative means (X-gal histochemistry and standard colourimetric enzyme assays, respectively). See Pickles, R. J., et al., 1996. Human Gene Therapy 7, 921-931.

While the invention has been described in connection with specific embodiments thereof, it will be understood that the invention is capable of further modification. This application is intended to encompass any variations, uses or adaptations of the invention that follow in general, the principles of the present invention and including such departures from the present disclosure as come within known, or customary practice within the art to which the invention pertains, as may be applied to the essential features set forth in the scope of the scope of the embodiment of the invention described above.

That which is claimed:

1. A method of delivering a heterologous nucleic acid into a cell, comprising:
contacting a conjugate to said cell, said conjugate comprising a transfer vector and a ligand, wherein said transfer vector comprises a heterologous nucleic acid to be delivered into said cell, and wherein said ligand specifically binds to a G protein-coupled receptor, and wherein said cell expresses said G protein-coupled receptor, under conditions that cause said vector to be internalized into said cell, and
wherein said ligand is selected from the group consisting of nucleotides represented by formulae I-III below, and their pharmaceutically acceptable salts: 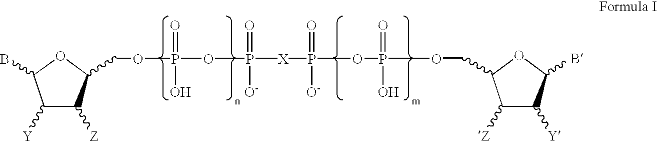
wherein:
X is oxygen, methylene, difluoromethylene, imido;
n=0, 1, or 2;
m=0, 1, or 2;
n+m=0, 1, 2, 3, or 4; and
B and B′ are each independently a purine residue or a pyrimidine residue linked through the 9- or 1-position, respectively;
Z=OH or N₃;
Z′=OH or N₃;
Y=H or OH;
Y′=H or OH;
provided that when Z is N₃, Y is H or when Z′ is N₃, Y′ is H; or 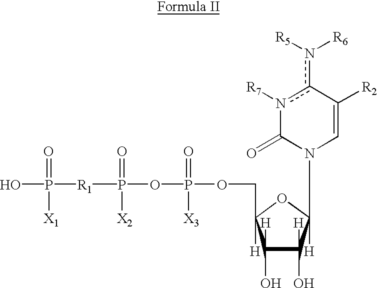
wherein:
R₁, X₁, X₂ and X₃ are each independently either O⁻or S⁻;
R₅ and R₆ are H while R₇ is nothing and there is a double bond between N-3 and C-4 (cytosine), or
R₅, R₆ and R₇ taken together are —CH═CH—, forming a ring from N-3 to N-4 with a double bond between N-4 and C-4 (3,N⁴-ethenocytosine) optionally substituted at the 4- or 5-position of the etheno ring; or 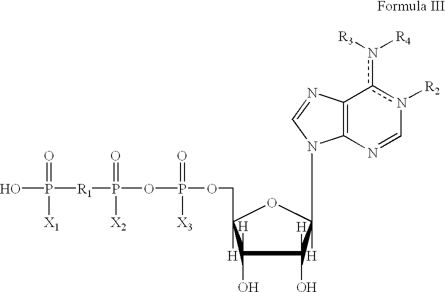
wherein:
R₁, X₁, X₂, and X₃ are defined as in Formula I;
R₃ and R₄ are H while R₂ is nothing and there is a double bond between N-1 and C-6 (adenine), or
R₃ and R₄ are H while R₂ is O and there is a double bond between N-1 and C-6 (adenine 1-oxide), or
R₃, R₄, and R₂ taken together are —CH═CH—, forming a ring from N-6 to N-1 with a double bond between N-6 and C-6 (1,N6-ethenoadenine) optionally substituted at the −4 or −5 position of the etheno ring;
or pharmaceutically acceptable esters or salts thereof.

2. The method of claim 1 wherein the compounds of Formula I are those of Formula Ia: 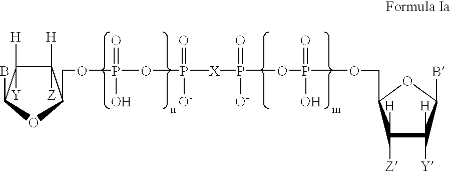
wherein:
X=O;
n+m=1 or 2;
Z, Z′, Y, and Y′=OH;
B and B′ are defined in Formulas Ib and Ic: 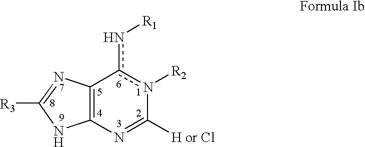
R₂ is O or is absent; or
R₁ and R₂ taken together may form optionally substituted 5-membered fused irnidazole ring; or
R₁ of the 6-HNR₁ group or R₃ of the 8-HNR₃ group is chosen from the group consisting of:
(a) arylalkyl (C₁₋₆) groups with the aryl moiety optionally substituted,
(b) alkyl,
(c) ([6-ainiohexyl]carbamoylmethyl),
(d) ω-ammo alkyl (C₂₋₁₀),
(e) ω-hydroxy alkyl (C₂₋₁₀),
(f) ω-thiol alkyl (C₂₋₁₀),
(g) ω-carboxy alkyl (C₂₋₁₀),
(h) the ω-acylated derivatives of (b), (c) or (d) wherein the acyl group is either acetyl, trifluroacetyl, benzoyl, or substituted-benzoyl alkyl(C₂₋₁₀), and
(i) ω-carboxy alkyl (C₂₋₁₀) as in (e) above wherein the carboxylic moiety is an ester or an amide; 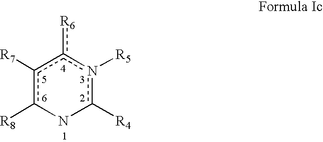
wherein:
R₄ is hydroxy, mercapto, amino, cyano, aralkoxy, C₁₋₆ alkylthio, C₁₋₆ alkoxy, C₁₋₆ alkylamino or dialkylanino, wherein the alkyl groups of said dialkylamino are optionally linked to form a heterocycle;
R₅ is hydrogen, acyl, C₁₋₆ allyl, aroyl, C₁₋₅ alkanoyl, benzoyl, or sulphonate;
R₆ is hydroxy, mercapto, alkoxy, aralkoxy, C₁₋₆-alkylthio, C₁₋₅ disubstituted amino, triazolyl, aikylamino or diallylamino, wherein the alkyl groups of said dialkylario are optionally linked to form a heterocycle or linked to N³ to form an optionally substituted ring;
R_{5-R}₆ together forms a 5 or 6-membered saturated or unsaturated ring bonded through N or O at R, wherein said ring is optionally substituted;
R₇ is selected from the group cons g of:
(a) hydrogen,
(b) hydroxy,
(c) cyano,
(d) nitro,
(e) alkenyl, wherein the alkenyl moiety is optionally linked through oxygen to form a ring optionally substituted with alkyl or aryl groups on the carbon adjacent to the oxygen,
(f) substituted allyyl
(g) halogen,
(h) alkyl,
(i) substituted alkyl,
(l) perhalomethyl,
(k) C₂₋₆ alyl,
(l) C₂₋₃ alkenyl,
(m) substituted ethenyl,
(n) C₂₋₃ alkynyl and
(o) substituted alkynyl when R₆ is other than amino or substituted amino;
R₈ is selected from the group consisting of:
(a) hydrogen,
(b) alkoxy,
(c) arylalkoxy,
(d) alkylthio,
(e) arylalkylthio,
(f) carboxamidomethyl,
(g) carboxymethyl,
(h) methoxy,
(i) methylthio,
(j) phenoxy and
(k) phenylthio.
wherein the substituted derivatives of adenine are adenine 1-oxide; 1,N6-(4- or 5-substituted etheno) adenine; 6-substituted adenine; or 8-substituted aminoadenine, where R′ of the 6- or 8-HNR′ groups are chosen from among:
arylalkyl (C₁₋₆) groups with the aryl moiety optionally functionalized; alkyl; and alkyl groups with functional groups therein, selected from the group consisting of ([6-aminohexyl]carbamoylmethyl)-, and ω-acylated-amino(hydroxy, thiol and carboxy) derivatives where the acyl group is acetyl, trifluroroacetyl, benzoyl or substituted-benzoyl and the carboxylic moiety is present as the ethyl or methyl ester derivative or the methyl, ethyl or benzamido derivative.

3. The method of claim 1 wherein the compounds of Formula I are those of Formula Ie: 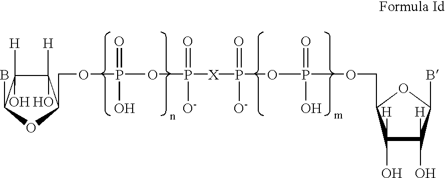
wherein:
X is oxygen, methylene, difluoromethylene, or imido;
n=0 or 1;
m=0 or 1;
n+m=0, 1, or 2; and
B and B′ are each independently a purine residue, as in Formula Ib as described in claim 2, or a pyrimidine residue, as in Formula Ic as described in claim 2, linked through the 9- or 1-position, respectively; provided that when B and B′ are uracil, attached at N-1 position to the ribosyl moiety, then the total of m+n equals 3 or 4 when X is oxygen.

4. The method of claim 1 wherein the furanose sugar of Formula I is in the β-D-configuration, or the D-configuration, or the L-configuration, or the D- and L-configuration.

5. A method according to claim 1, wherein said vector is a viral vector.

6. A method according to claim 1, wherein said vector is a viral vector selected from the group consisting of adenovirus vectors, adeno-associated virus vectors, human retrovirus vectors, nonhuman retrovirus vectors, and herpes virus vectors.

7. A method according to claim 7, wherein said viral vector is selected from the group consisting of lentivirus vectors and Moloney Murine Leukemia virus vectors.

8. A method according to claim 1, wherein said vector is an oligonucleotide.

9. A method according to claim 1, wherein said ligand is an antibody.

10. A method according to claim 1, wherein said ligand is a peptide.

11. A method according to claim 1, wherein said ligand is selected from the group consisting of nucleotides, nucleosides, catecholamines, C5A, and bradykinin.

12. A method according to claim 1, wherein said ligand is selected from the group consisting of G protein-coupled receptor agonists and G protein-coupled receptor antagonists.

13. A method according to claim 1, wherein said conjugate is a covalent conjugate.

14. A method according to claim 1, wherein said cell is an airway epithelial cell.

15. A method according to claim 1, wherein said cell is a differentiated columnar airway epithelial cell.

16. A method according to claim 1, wherein said contacting step is carried out in vitro.

17. A method according to claim 1, wherein said contacting step is carried out in vivo.

18. A method according to claim 1, wherein said conjugate is formed prior to said contacting step.

19. A bispecific antibody having a first combining region that specifically binds to a viral vector and a second combining region that specifically binds to an extracellular epitope of a G protein-coupled receptor.

20. A conjugate useful for delivering a heterologous nucleic acid into a cell, said conjugate comprising a transfer vector and a ligand, wherein said transfer vector comprises a heterologous nucleic acid to be delivered into said cell, and wherein said ligand specifically binds to a G protein-coupled receptor., and wherein said ligand is selected from the group consisting of nucleotides represented by formulae I-III, claim 1.

21. A conjugate according to claim 20, wherein said vector is a viral vector.

22. A conjugate according to claim 20, wherein said vector is a viral vector selected from the group consisting of adenovirus vectors, adeno-associated virus vectors, human retrovirus retrovirus vectors, nonhuman retrovirus vectors, and herpes virus vectors.

23. A conjugate according to claim 20, wherein said vector is a viral vector selected from the group consisting of lentivirus vectors and Moloney Murine Leukemia Virus vectors.

24. A conjugate according to claim 20, wherein said ligand is an antibody.

25. A conjugate according to claim 20, wherein said ligand is a peptide.

26. A conjugate according to claim 20, wherein said ligand is selected from the group consisting of nucleotides, nucleosides, catecholamines, C5A, and bradykinin.

27. A conjugate according to claim 20, wherein said ligand is selected from the group consisting of G protein-coupled receptor agonists and G protein-coupled receptor antagonists.

28. A conjugate according to claim 20, wherein said conjugate is a covalent conjugate.
